SEQUENCE LISTING (61 sequences)

SEQ ID NO: 1 - protein, 410 aa, mus musculus
MGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKST

SEQ ID NO: 2 - protein, 430 aa, mus musculus
MGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 3 - protein, 451 aa, mus musculus
MGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTEGEVNAEEEGFENLWTTASTFIVLFLLSLFYSTTVTLFKVK

SEQ ID NO: 4 - protein, 284 aa, mus musculus
MFLLSECKAPEENEKINLGCLVIGSQPLKISWEPKKSSIVEHVFPSEMRNGNYTMVLQVTVLASELNLNHTCTINKPKRKEKPFKFPESWDSQSSKRVTPTLQAKNHSTEATKAITTKKDIEGAMAPSNLTVNILTTSTHPEMSSWLLCEVSGFFPENIHLMWLGVHSKMKSTNFVTANPTAQPGGTFQTWSVLRLPVALSSSLDTYTCVVEHEASKTKLNASKSLAISGIVNTIQHSCIMDEQSDSYMDLEEENGLWPTMCTFVALFLLTLLYSGFVTFIKVK

SEQ ID NO: 5 - protein, 279 aa, mus musculus
MFLLSECKAPEENEKINLGCLVIGSQPLKISWEPKKSSIVEHVFPSEMRNGNYTMVLQVTVLASELNLNHTCTINKPKRKEKPFKFPESWDSQSSKRVTPTLQAKNHSTEATKAITTKKDIEGAMAPSNLTVNILTTSTHPEMSSWLLCEVSGFFPENIHLMWLGVHSKMKSTNFVTANPTAQPGGTFQTWSVLRLPVALSSSLDTYTCVVEHEASKTKLNASKSLAISGKSQLGKSVNQGQHLVPMIDKYSCLGRGGLHCLDKRNTVLICFSLKDRTT

SEQ ID NO: 6 - protein, 251 aa, mus musculus
MFLLSECKAPEENEKINLGCLVIGSQPLKISWEPKKSSIVEHVFPSEMRNGNYTMVLQVTVLASELNLNHTCTINKPKRKEKPFKFPESWDSQSSKRVTPTLQAKNHSTEATKAITTKKDIEGAMAPSNLTVNILTTSTHPEMSSWLLCEVSGFFPENIHLMWLGVHSKMKSTNFVTANPTAQPGGTFQTWSVLRLPVALSSSLDTYTCVVEHEASKTKLNASKSLAISGCYHLLPESDGPSRRPDGPALA

SEQ ID NO: 7 - protein, 375 aa, homo sapiens [sequence omitted]

SEQ ID NO: 8 - protein, 78 aa, home sapiens
GSASAPTLFPLVSCENSPSDTSSVAVGCLAQDFLPDSITFSWKYKNNSDISSTRGFPSVLRGGKYAATSQVLLPSKDV

SEQ ID NO: 9 - DNA, 1378 bp, mus musculus
attctaaaggggtctatgatagtgtgactactttgactactggggccaaggcaccactctcacagtctcctcagagagtcagtccttcccaaatgtcttccccctcgtctcctgcgagagccccctgtctgataagaatctggtggccatgggctgcctagcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccact

SEQ ID NO: 10 - DNA, 1441 bp, mus musculus
attctaaaggggtctatgatagtgtgactactttgactactggggccaaggcaccactctcacagtctcctcagagagtcagtccttcccaaatgtcttccccctcgtctcctgcgagagccccctgtctgataagaatctggtggccatgggctgcctagcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattga

SEQ ID NO: 11 - DNA, 1504 bp, mus musculus
attctaaaggggtctatgatagtgtgactactttgactactggggccaaggcaccactctcacagtctcctcagagagtcagtccttcccaaatgtcttccccctcgtctcctgcgagagccccctgtctgataagaatctggtggccatgggctgcctagcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactgagggggaggtgaatgctgaggaggaaggctttgagaacctgtggaccactgcctccaccttcatcgtcctcttcctcctgagcctcttctacagcaccaccgtcaccctgttcaaggtgaaatga

SEQ ID NO: 12 - DNA, 1270 bp, mus musculus
aaaaaagaatggtatcaaaggacagtgcttagatccaaggtgataaaaaggaacctgacatgttcctcctctcagagtgcaaagccccagaggaaaatgaaaagataaacctgggctgtttagtaattggaagtcagccactgaaaatcagctgggagccaaagaagtcaagtatagttgaacatgtcttcccctctgaaatgagaaatggcaattatacaatggtcctccaggtcactgtgctggcctcagaactgaacctcaaccacacttgcaccataaataaacccaaaaggaaagaaaaacctttcaagtttcctgagtcatgggattcccagtcctctaagagagtcactccaactctccaagcaaagaatcactccacagaagccaccaaagctattaccaccaaaaaggacatagaaggggccatggcacccagcaacctcactgtgaacatcctgaccacatccacccatcctgagatgtcatcttggctcctgtgtgaagtatctggcttcttcccggaaaatatccacctcatgtggctgggtgtccacagtaaaatgaagtctacaaactttgtcactgcaaaccccaccgcccagcctgggggcacattccagacctggagtgtcctgagactaccagtcgctctgagctcatcacttgacacttacacatgtgtggtggaacatgaggcctcaaagacaaagcttaatgccagcaagagcctagcaattagtggcatagtcaacaccatccaacactcgtgtatcatggatgagcaaagtgacagctacatggacttagaggaggagaacggcctgtggcccacaatgtgcaccttcgtggccctcttcctgctcacactgctctacagtggcttcgtcaccttcatcaaggtgaagtagaccaggacagcagaatcctgcaactacagagaaaagtgctttccctcaacatgaagccaactaagaagataccttctatgcagagagaaatgccaaggccctcctctcaatacctgctctccacctagactcccagactcaaaatgcctagtatccttggctataggcaaagcaggtgttctgctgctccagccctactcatcgatgtcccctgcctccccccaagccctcatcattccataagcatgctgtggtctaccccacctgcctcctggtaatttgtttgtttgtttgtttgtttgtttctgtgatctaaataaactcaaaccacatgagatctgaaaaaaaaaaaaaaa

SEQ ID NO: 13 - DNA, 1412 bp, mus musculus
aaaaaagaatggtatcaaaggacagtgcttagatccaaggtgataaaaaggaacctgacatgttcctcctctcagagtgcaaagccccagaggaaaatgaaaagataaacctgggctgtttagtaattggaagtcagccactgaaaatcagctgggagccaaagaagtcaagtatagttgaacatgtcttcccctctgaaatgagaaatggcaattatacaatggtcctccaggtcactgtgctggcctcagaactgaacctcaaccacacttgcaccataaataaacccaaaaggaaagaaaaacctttcaagtttcctgagtcatgggattcccagtcctctaagagagtcactccaactctccaagcaaagaatcactccacagaagccaccaaagctattaccaccaaaaaggacatagaaggggccatggcacccagcaacctcactgtgaacatcctgaccacatccacccatcctgagatgtcatcttggctcctgtgtgaagtatctggcttcttcccggaaaatatccacctcatgtggctgggtgtccacagtaaaatgaagtctacaaactttgtcactgcaaaccccaccgcccagcctgggggcacattccagacctggagtgtcctgagactaccagtcgctctgagctcatcacttgacacttacacatgtgtggtggaacatgaggcctcaaagacaaagcttaatgccagcaagagcctagcaattagtggtaagtcacaactgggtaagagtgtcaatcaaggacagcacttggtacctatgatagacaaatactcctgtttgggaagaggaggtctgcattgtctagataagaggaacactgtgcttatctgtttcagtttaaaagacagaactacataacacttcaccctttctacaacacttagatttttcagccctctcctctatggtgtttcctgaaccctgaagaaagtgatatagatgtttattttctagggccactgttccaaagaaacaatcacgtattattagcagcttgaccaggtgtgaatttcccccagtatccacttctgctccctgtgggatgaagtccctccataagtaaagttgaaggctgtgggcataaatgcaaatgcctgtaatccaattttacaagtccatgacatacatttaacaaacaatagcagttacttctctactggagcctatgaactccccagctataggtttttcccttctgcagagcagagcttaaatccaatagggagggagttggttgtacccataactgccataccactattacaccaataagcacatcttacatagcaagacaattagtgtagcactcaaagtccatgttcaagagagaatgttggcaacagcctatataacatctcccagcacagaaaa

SEQ ID NO: 14 - DNA, 1012 bp, mus musculus
aaaaaagaatggtatcaaaggacagtgcttagatccaaggtgataaaaaggaacctgacatgttcctcctctcagagtgcaaagccccagaggaaaatgaaaagataaacctgggctgtttagtaattggaagtcagccactgaaaatcagctgggagccaaagaagtcaagtatagttgaacatgtcttcccctctgaaatgagaaatggcaattatacaatggtcctccaggtcactgtgctggcctcagaactgaacctcaaccacacttgcaccataaataaacccaaaaggaaagaaaaacctttcaagtttcctgagtcatgggattcccagtcctctaagagagtcactccaactctccaagcaaagaatcactccacagaagccaccaaagctattaccaccaaaaaggacatagaaggggccatggcacccagcaacctcactgtgaacatcctgaccacatccacccatcctgagatgtcatcttggctcctgtgtgaagtatctggcttcttcccggaaaatatccacctcatgtggctgggtgtccacagtaaaatgaagtctacaaactttgtcactgcaaaccccaccgcccagcctgggggcacattccagacctggagtgtcctgagactaccagtcgctctgagctcatcacttgacacttacacatgtgtggtggaacatgaggcctcaaagacaaagcttaatgccagcaagagcctagcaattagtggatgctaccacctcctgcctgagtcagacggtccttccaggagacctgatggtcctgcccttgcctgagacctttctaggctgaatggtcatcatgtccactgtctggttagcttggttgcctctgtgttaagttgcttcatactacatgtagcaaggaaagttacctttccacttctcaggacactgtaaagaagcacttctgtaaattatagtgagaaacgatcaataataaatgaaatgaacaaacataaaaaaaaaaaaaa

SEQ ID NO: 15 - DNA, 1866 bp, mus musculus
aaaaaagaatggtatcaaaggacagtgcttagatccaaggtgataaaaaggaacctgacatgttcctcctctcagagtgcaaagccccagaggaaaatgaaaagataaacctgggctgtttagtaattggaagtcagccactgaaaatcagctgggagccaaagaagtcaagtatagttgaacatgtcttcccctctgaaatgagaaatggcaattatacaatggtcctccaggtcactgtgctggcctcagaactgaacctcaaccacacttgcaccataaataaacccaaaaggaaagaaaaacctttcaagtttcctgagtcatgggattcccagtcctctaagagagtcactccaactctccaagcaaagaatcactccacagaagccaccaaagctattaccaccaaaaaggacatagaaggggccatggcacccagcaacctcactgtgaacatcctgaccacatccacccatcctgagatgtcatcttggctcctgtgtgaagtatctggcttcttcccggaaaatatccacctcatgtggctgggtgtccacagtaaaatgaagtctacaaactttgtcactgcaaaccccaccgcccagcctgggggcacattccagacctggagtgtcctgagactaccagtcgctctgagctcatcacttgacacttacacatgtgtggtggaacatgaggcctcaaagacaaagcttaatgccagcaagagcctagcaattagtggtaagtcacaactgggtaagagtgtcaatcaaggacagcacttggtacctatgatagacaaatactcctgtttgggaagaggaggtctgcattgtctagataagaggaacactgtgcttatctgtttcagtttaaaagacagaactacataacacttcaccctttctacaacacttagatttttcagccctctcctctatggtgtttcctgaaccctgaagaaagtgatatagatgtttattttctagggccactgttccaaagaaacaatcacgtattattagcagcttgaccaggtgtgaatttcccccagtatccacttctgctccctgtgggatgaagtccctccataagtaaagttgaaggctgtgggcataaatgcaaatgcctgtaatccaattttacaagtccatgacatacatttaacaaacaatagcagttacttctctactggagcctatgaactccccagctataggtttttcccttctgcagagcagagcttaaatggaaaacagtggagaagccactagggaggggtatcagagtcacactgaagaaaattgttcagtgaactaagaaactctcctcccaccttaaactctcagttgacttctcacatacacataagcatatacacatgcacatgtgcacatcacagtcacatgcatgataacacaggcagacgtgcatgagtgcacatgcatacctactcagatgcacataaatttacagcagagccttaggtgacaggaatggggactgtggtcatttgaataagaaccccataggctcatatagttgaatgcttagtcaccagggagtggaactctttaacaggatcagaaagattcagaggcaagtccttgttgtaggaagtgtgtgtcactggagcacgggtaggctttcaggtttcaaaagttcatgtcaggactagagtctctctgtcactctgtgtctctatatctcttctgaattctaggtaatttctccagcactatgtccactggcataccaccatgctccctaccatgatgtaaatgaactaacccctgaaactgttagcaagcctcaattaaatgctttcttttataagaaaaaaaaaaaaaaaaaaaaaaa

SEQ ID NO: 16 - DNA, 1918 bp, homo sapiens
tgagtgtgtgcagcacctacgtgctgatgcctcgggggaaagcaggcctggtccacccaaacctgagccctcagccattctgagcagggagccaggggcagtcaggcctcagagtgcagcagggcagccagctgaatggtggcagggatggctcagcctgctccaggagaccccaggtctgtccaggtgttcagtgctgggccctgcagcaggatgggccgaggcctgcagccccagcagccttggacaaagacctgaggcctcaccacggccccgccacccctgatagccatgacagtctgggctttggaggcctgcaggtgggctcggccttggtggggcagccacagcgggacgcaagtagtgagggcactcagaacgccactcagccccgacaggcagggcacgaggaggcagctcctcaccctccctttctcttttgtcctgcgggtcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgccgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtggccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccggtaaacccaccctgtacaacgtgtccctggtcatgtccgacacagctggcacctgctactgaccctgctggcctgcccacaggctcggggcggctggccgctctgtgtgtgcatgcaaactaaccgtgtcaacggggtgagatgttgcatcttataaaatt

SEQ ID NO: 17 - DNA, 233 bp, homo sapiens
gggagtgcatccgcccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtc

SEQ ID NO: 18 - DNA, 27 bp, mus muculus
ttctaaaggggtctatgatagtgtgac

SEQ ID NO: 19 - DNA, 39 bp, mus musculus
aaagaatggtatcaaaggacagtgcttagatccaaggtg

SEQ ID NO: 20 - DNA, 31 bp, Artificial Sequence
  primer
ccggaattcggctgcctagcccgggacttcc

SEQ ID NO: 21 - DNA, 33 bp, Artificial Sequence
  primer
cggctcgagtcaatagcaggtgccgcctgtgtc

SEQ ID NO: 22 - DNA, 31 bp, Artificial Sequence; identical to SEQ ID NO: 20
  primer

SEQ ID NO: 23 - DNA, 34 bp, Artificial Sequence
  primer
cggctcgagtcatttcaccttgaacagggtgacg

SEQ ID NO: 24 - DNA, 26 bp, Artificial Sequence
  primer
cacggcaggtgtacacattcaggttc

SEQ ID NO: 25 - DNA, 28 bp, Artificial Sequence
  primer
cgtggcctcgcagatgagtttagacttg

SEQ ID NO: 26 - DNA, 21 bp, Artificial Sequence
  primer
ggatgttcacagtgaggttgc

SEQ ID NO: 27 - DNA, 20 bp, Artificial Sequence
  primer
agtgacctggaggaccattg

SEQ ID NO: 28 - DNA, 23 bp, Artificial Sequence
  primer
gcaacctcactgtgaacatcctg

SEQ ID NO: 29 - DNA, 23 bp, Artificial Sequence
  primer
gcttaatgccagcaagagcctag

SEQ ID NO: 30 - DNA, 20 bp, Artificial Sequence
  primer
taggttcagttgctcacgag

SEQ ID NO: 31 - DNA, 19 bp, Artificial Sequence
  primer
tgaccatcgagaacaaagg

SEQ ID NO: 32 - DNA, 21 bp, Artificial Sequence
  primer
ctcctctcagagtgcaaagcc

SEQ ID NO: 33 - DNA, 21 bp, Artificial Sequence
  primer
ggatgttcacagtgaggttgc

SEQ ID NO: 34 - DNA, 22 bp, Artificial Sequence
  primer
catgagcagccagttaaccctg

SEQ ID NO: 35 - DNA, 21 bp, Artificial Sequence
  primer
atgcagccatcgcaccagcac

SEQ ID NO: 36 - DNA, 22 bp, Artificial Sequence
  primer
gactccctgaacatgagcactg

SEQ ID NO: 37 - DNA, 22 bp, Artificial Sequence
  primer
ggtactgtgctggctgtttgag

SEQ ID NO: 38 - DNA, 24 bp, Artificial Sequence
  primer
ctggagtctgacctctacactctg

SEQ ID NO: 39 - DNA, 24 bp, Artificial Sequence
  primer
caggtcagactgactttatccttg

SEQ ID NO: 40 - DNA, 22 bp, Artificial Sequence
  primer
gatgtctgtgctgaggcccagg

SEQ ID NO: 41 - DNA, 22 bp, Artificial Sequence
  primer
ggaagctcttctgatcccagag

SEQ ID NO: 42 - DNA, 24 bp, Artificial Sequence
  primer
gagtcagtgactgtgacttggaac

SEQ ID NO: 43 - DNA, 20 bp, Artificial Sequence
  primer
accaggcaagtgagactgac

SEQ ID NO: 44 - DNA, 20 bp, Artificial Sequence
  primer
ctggctgcagtgacacatct

SEQ ID NO: 45 - DNA, 20 bp, Artificial Sequence
  primer
ggtggttatggagagcctca

SEQ ID NO: 46 - DNA, 20 bp, Artificial Sequence
  primer
tggggtttggctacacagat

SEQ ID NO: 47 - DNA, 20 bp, Artificial Sequence
  primer
cccaccaccaaagacatacc

SEQ ID NO: 48 - DNA, 20 bp, Artificial Sequence
  primer
gtaccctgagcaacgaccat

SEQ ID NO: 49 - DNA, 20 bp, Artificial Sequence
  primer
gtaccctgagcaacgaccat

SEQ ID NO: 50 - DNA, 20 bp, Artificial Sequence
  primer
tgcctctcctcctcttcttg

SEQ ID NO: 51 - DNA, 20 bp, Artificial Sequence
  primer
tgatgatgcggttcttggta

SEQ ID NO: 52 - DNA, 21 bp, Artificial Sequence
  primer
tcagaagagggacgcattgtg

SEQ ID NO: 53 - DNA, 21 bp, Artificial Sequence
  primer
ttcaagccctcataggtgtga

SEQ ID NO: 54 - DNA, 21 bp, Artificial Sequence
  primer
cttgcagatctagtcagagcc

SEQ ID NO: 55 - DNA, 23 bp, Artificial Sequence
  primer
caatgggtgaagttgatgtcttg

SEQ ID NO: 56 - DNA, 22 bp, Artificial Sequence
  primer
ccaagtcttcgccatcagtcac

SEQ ID NO: 57 - DNA, 22 bp, Artificial Sequence
  primer
gaacagtcagcacgggacaaac

SEQ ID NO: 58 - DNA, 23 bp, Artificial Sequence
  primer
  misc_feature (1)..(1): s is c or g
  misc_feature (3)..(3): r is a or g
  misc_feature (6)..(6): n is a, c, g, or t
  misc_feature (7)..(7): m is a or c
  misc_feature (13)..(13): s is c or g
  misc_feature (16)..(16): s is c or g
  misc_feature (21)..(21): w is a or t
sargtnmagctgsagsagtcwgg

SEQ ID NO: 59 - DNA, 18 bp, Artificial Sequence
  primer
atagcaggtgtccactcc

SEQ ID NO: 60 - DNA, 21 bp, Artificial Sequence
  primer
caaagtgaccccttacctgct

SEQ ID NO: 61 - DNA, 20 bp, Artificial Sequence
  primer
ctgacattggaggtgtgtgt

(19) United States
(12) Patent Application Publication
(10) Pub. No.: US 2007/0196365 A1
(43) Pub. Date: Aug. 23, 2007

(54) Immunoglobulin heavy chain variants expressed in mesenchymal cells and therapeutic uses thereof

(76) Inventors: Dov Zipori, Rehovot (IL); Yaron Shav-Tal, Elkanah (IL); Mira Barda-Saad, Ganei Tikva (IL); Smadar Lapter, Tel-Aviv (IL)

Correspondence Address: WINSTON & STRAWN LLP;PATENT DEPARTMENT, 1700 K STREET, N.W., WASHINGTON, DC, 20006, US

(21) Appl. No.: 11/657,211

(22) Filed: Jan. 23, 2007

Related U.S. Application Data
(63) Continuation-in-part of application No. 10/643,982, filed on Aug. 20, 2003, now abandoned.
Application No. 10/643,982 is a continuation of application No. PCT/IL02/00129, filed on Feb. 20, 2002.
(60) Provisional application No. 60/859,928, filed on Nov. 20, 2006.

(30) Foreign Application Priority Data
Feb. 20, 2001 (IL) 141539
Sep. 25, 2001 (IL) 145658

(51) Int. Cl. A61K 39/395; C07H 21/02; C07H 21/04; C12P 21/06; C12N 5/06; C07K 16/30

(52) U.S. Cl. 424/133.1; 435/69.1; 435/320.1; 435/326; 530/388.1; 530/350; 536/23.5; 536/23.1; 424/155.1

(57) ABSTRACT

Mesenchymal cells are unexpectedly found to express specific truncated versions of immunoglobulin (Ig) superfamily members, Igμ heavy chain and Igδ heavy chain variants. Mesenchymal Ig heavy chain gene products either directly or indirectly control hemopoietic stem cells. Ectopic expression, RNAi or antibody therapy can be used to modulate Ig heavy chain mediated functions.

### CROSS REFERENCE TO RELATED APPLICATIONS

This application is a continuation-in-part of U.S. application Ser. No. 10/643,982 filed Aug. 20, 2003, which is a continuation of International Application No. PCT/IL02/00129 filed Feb. 20, 2002; and this application claims the benefit of U.S. Provisional Application No. 60/859,928 filed Nov. 20, 2006. The entire content of each mentioned application is expressly incorporated herein by reference thereto.

### FIELD OF THE INVENTION

The present invention relates to isolated truncated immunoglobulin heavy chain polypeptide variants expressed in mesenchymal stem cells, in particular Cμ and Cδ, compositions comprising same and methods of use thereof. In various embodiments the variants are useful in inhibiting aberrant cell growth and proliferation.

### BACKGROUND OF THE INVENTION

The pre B Cell Receptor (preBCR)

In the bone marrow, B cell development can be divided into different stages, based on the rearrangement status of the IgH and IgL chain loci (Ehlich et al 1994; ten Boekel et al 1997) and the expression of intracellular and surface-bound markers. The pre-B cell receptor consists of immunoglobulin μ heavy chains and surrogate light chain, the VpreB and λ5 proteins (Hardy et al 1991).

Immunoglobulins (Igs) are synthesized exclusively by B lymphocytes (Abbas et al 1994). The immunoglobulin molecule can exist in two very different environments: at the cell membrane as a surface antigen receptor and in solution as a secreted antibody. The immunoglobulin molecule is composed of two identical light chains and two identical heavy chains. The light and heavy chains can each be divided into an N terminal variable (V) and a C terminal constant (C) region. The V regions are responsible for antigen binding, whereas the C regions embody the various effector functions of the molecule. The various classes of immunoglobulins with different functions (IgM, IgD, IgG, IgA, IgE) are distinguished by different heavy chains (μ, δ, λ, α, ε), with the difference residing in their C_{H} regions (Cμ, Cδ, Cλ Cα, Cε) (Rogers et al 1980).

B lymphocytes mature from hemopoietic stem cells through a series of developmental stages that are characterized by sequential DNA rearrangements of Ig gene segments. The rearrangement of Ig genes allows B cells to respond to a wide spectrum of foreign antigens (Ags). The V, D and J segments encoding parts of the IgH and the V and J segments of IgL-chains are rearranged in a stepwise fashion (Melchers, & Rolink 1999). ProB cells begin to rearrange D_{H} to J_{H} segments of the H chain locus, so that in PreBI cells (B220⁺, c-kit⁺) both H-chain alleles are D_{H}J_{H} rearranged. ProB and PreBI cells already produce surrogate light chains VpreB and λ5 in preparation for the formation of the preBCR (Melchers et al 1993). When V_{H} to D_{H} to J_{H} rearrangements are initiated in PreBI cells, those rearrangements that are in frame will generate a functional IgH chain gene.

The formation of the preBCR has a functional consequence for precursor B cells. PreBII cells are stimulated to undergo between two and five rounds of divisions (Rolink et al 2000) and to expand the number of μH chain producing preBII cells in which, subsequently, L-chain rearrangements are initiated. The preBCR signals for the inhibition of rearrangements at the second D_{H} J_{H}—rearranged H chain allele (allelic exclusion) (Ehlich et al 1994; ten Boekel et al 1997). Subsequent processing of the RNA leads to splicing out of the intron between the VDJ complex and the most proximal C region gene, which is the C—giving rise to a functional mRNA for the μ heavy chain.

The recombination activating genes, RAG-1 and RAG-2, are essential for V(D)J recombination (Shinkai et al 1992, Mombaerts et al 1992). During B lineage development in adult mice, RAG-1 and RAG-2 are expressed exclusively in early B progenitors of the bone marrow and expression ceases prior to the migration of B lineage cells from the bone marrow (Hardy et al 1991; Osmond 1990). Furthermore, mice that lack either RAG-1 or RAG-2 fail to develop mature lymphocytes due to their inability to initiate rearrangement of the antigen receptor genes (Shinkai et al 1992; Mombaerts et al 1992). However, expression of a rearranged μHC transgene in the RAG-deficient background partially rescued this developmental block in the B lineage, leading to the generation of B220⁺CD43⁻ pre-B cells, demonstrating that μ chain expression was sufficient to drive this developmental transition (Young et al 1994, Spanopoulou et al 1994).

Mu (μ) chains of membrane (μₘ) and secreted (μₛ) forms differ in structure. The μₘ chain is larger than the μₛ chain and has hydrophobic properties not exhibited by the μₛ (Rogers et al 1980). An essential role for components of the preBCR complex has been established. Targeted disruption of the membrane exons of the μH chain, or the λ5 locus, result in the failure of normal B cell development and the loss of allelic exclusion in pre-B cells (Kitamura et al 1991; Kitamura et al 1992a; Kitamura et al 1992b; Loffert et al 1996). PreB cells can express μₛ chains as well as, μₘ chains providing a potential source for a soluble form of preBCR. The μₛ chains can associate with SLC and assemble into a soluble preBCR complex in preB cells. μₛ chains can associate with SLC internally, but are efficiently retained and degraded. Mutation of a single cysteine (Cys575) in the μₛ tailpiece (tp) results in the release of soluble preBCR from the endoplasmic reticulum (ER) and its subsequent secretion.

The soluble preBCR does not bind the hapten recognized by antibody (Ab) consisting of the same heavy chain V region paired with a conventional L chain, consistent with the preBCR having a unique specificity (Bornemann et al 1997).

Because the preBCR, like the mature BCR, has no known intrinsic enzymatic functions, it must rely upon associated proteins to provide a functional linkage with intracellular signaling pathways. The mature and preBCR—associated Igα and Igβ chains contain immunoreceptor tyrosine-based activation motifs (ITAMs), which are targets for phosphorylation by tyrosine kinases (Reth 1984); these proteins are required for normal B cell development (Gong & Nussenzweig 1996; Torres et al 1996). Furthermore, the importance of an ITAM-associated tyrosine kinase activity during early B lymphopoiesis was demonstrated in mice deficient in the syk tyrosine kinase, in which an incomplete block in development was observed at the B220⁺ CD43⁺proB cell stage (Cheng, et al 1995; Turner et al 1995).

Truncated heavy chain Dμ

Reth and Alt discovered (Reth and Alt, 1984) a truncated Dμ heavy chain in a permanent lymphoid cell line, which represents a pre B stage of B-lymphocytes, by transformation of bone marrow or fetal calf liver cells with Abelson murine leukemia virus (A-MuLV). Some A-MuLV generated lines produce an unusually small μ heavy chain mRNA and sometimes a small μ protein. The short μ mRNA sequences arise from the transcription of DJ_{H} rearrangements and the short μ proteins from the translation of the resulting DJ_{H} Cμ containing mRNAs (Dμ mRNA). Due to an inexact joining mechanism, the D_{H} can be rearranged to the J_{H} in three possible reading frames (RFs). A majority of the D_{H} segments carry their own promoter and an ATG translational initiation codon. When the D_{H} is rearranged to a J_{H} in RF2, according to the nomenclature of. (Ichihara et al 1989), this D_{H}J_{H} complex can be translated into a truncated μ chain protein. The size of these small μ chains was analyzed by Western blot using anti-IgM antisera and ¹²⁵I-labelled monoclonal IgM antibody. Lysates from control transformant express normal-sized μ-chains of 70 Kd molecular weight while cell lines express an abnormally small μ protein of approximately 57 Kd. Furthermore, instead of normal 2.4 and 2.7 kb μ mRNAs which encode, respectively, the secreted and membrane-bound forms of the μ proteins, cell lines 300-19 and 298-13 (Reth and Alt 1984) contain truncated Cμ-specific RNAs of 2.0 and 2.3 kb; these species contain 3′ ends specific to the membrane and secreted forms of the protein, respectively. Dμ preBCR can mediate a block in B cell development, probably by inhibiting V_{H} to D_{H}J_{H} rearrangements, as well as inducing V_{L} to J_{L} rearrangements (Tornberg et al 1998, Horne et al 1996).

International Patent Application Publication Nos. WO 02/066648 and WO02/066636, to some of the inventors of the present application, teach novel truncated transcripts of immunoglobulin superfamily genes, particularly Ig heavy chain variants and T cell receptor variants, respectively.

There is an unmet need for and it would be advantageous to have polypeptide or peptide markers for mesenchymal cells that are involved in control of proliferation and differentiation of hemopoietic stem cells. In addition, it would be advantageous to develop interventive therapeutic strategies based either on gene therapy or antisense molecular therapy to treat disorders involving the proliferation and differentiation of hemopoietic stem cells.

### SUMMARY OF THE INVENTION

The present invention relates to isolated B cell receptor polypeptides expressed in mesenchymal stem cells, polynucleotides encoding same and methods of use thereof. The present invention is based in part on the unexpected discovery of immunoglobulin (Ig) heavy chain (HC) mRNA encoding truncated Ig heavy chain polypeptides in early embryo and adult mesenchymal stem cells (MSC). Additionally, the unexpected showing that Igδ HC substitutes for Igμ HC in the oocyte, morula, mesenchyme of the early embryo, as well as in the adult mesenchyme in Ig μ chain deficient mice finding implies a role for Ig gene products in the regulation of early embryogenesis and in MSC functions. The ectopic expression of a mesenchymal truncated μ heavy chain in 293T cells resulted in G1 growth arrest.

It is an object of the present invention to provide polypeptide or peptide markers for mesenchymal stem cells that are involved in regulating proliferation and differentiation. It is another object of the present invention to provide methods for therapeutic intervention utilizing methods of gene therapy to treat disorders involving aberrant proliferation and differentiation.

The present invention discloses novel transcripts of Immunoglobulin (Ig) superfamily genes, in particular truncated Ig heavy chain variants, expressed by mesenchymal cells which are mediators of intercellular interactions leading, either directly or indirectly, to modulation in the proliferation and differentiation.

More preferably, the Ig variants are either directly or indirectly involved in the regulation of stem cell growth and differentiation. The therapeutic uses of these molecules are also disclosed.

The growth and differentiation of normal cells and malignant tumors within different tissue types, are dependent on mesenchymal cellular interactions, as is known in the art.

The present invention relates, in one aspect, to isolated polynucleotide molecules transcribed by immunoglobulin genes, said polynucleotide molecules lacking V (variant) regions and comprising a constant (C) domain and a 5′ intronic upstream sequence. The novel polynucleotides of the invention are exemplified herein by truncated transcripts of Ig μ and Ig δ chains.

The novel polynucleotide sequences disclosed herein and the corresponding proteins, polypeptides or peptides encoded by these polynucleotide sequences may be derived from any mammalian species including human genetic material.

In some embodiments the polynucleotide molecules lack V (variant) and D (diversity) regions.

In one embodiment of the present invention, the polynucleotide molecules comprise a cDNA molecule of a transcript consisting of a constant (Cμ) domain, and a 5′ intronic upstream sequence further comprising a 5′ joining (J) region domain. In some embodiments the polynucleotide molecules further comprise a 3′ nucleotide sequences encoding a secretory domain and a transmembrane domain. In various embodiments the polynucleotide of the present invention is selected from a polynucleotide set forth in any one of SEQ ID NOS: 9-11, SEQ ID NOS: 16-17 or a fragment thereof.

In another embodiment of the present invention, the polynucleotide molecules comprise a cDNA molecule of a transcript consisting of a constant (Cδ) domain and a 5′ intronic upstream sequence. In some embodiments the polynucleotide molecules further comprise a 3′ nucleotide sequences encoding a secretory domain and a transmembrane domain. In various embodiments the polynucleotide of the present invention is selected from a polynucleotide set forth in any one of SEQ ID NOS: 12-15 or a fragment thereof.

In another aspect, the invention relates to antisense and siRNA nucleic acid molecules of the polynucleotide molecules of the invention described hereinabove.

The invention further relates to expression vectors comprising the polynucleotide molecules of the present invention including antisense and siRNA nucleic acid molecules of the invention, and to host cells, particularly mammalian cells, comprising said vectors.

In another aspect the present invention relates to isolated truncated Ig heavy chain polypeptides said polypeptides molecules lacking V regions and comprising a constant (C) domain. In some embodiments the polypeptide molecules lack V (variant) and D (diversity) regions.

The novel polynucleotides of the invention are exemplified herein by truncated polypeptides of Ig μ and Ig δ chains.

In some embodiments of the invention, the cDNA molecule encodes a truncated μ heavy chain polypeptide having an amino acid sequence set forth in any one of SEQ ID NOS: 1-3 or 7-8, or a fragment thereof.

In another embodiment of the invention, the cDNA molecule encodes a truncated δ heavy chain polypeptide having an amino acid sequence set forth in any one of SEQ ID NOS: 4-6, or a fragment thereof.

In yet another aspect the present invention provides a pharmaceutical composition comprising as an active agent the nucleic acid molecules or the polypeptides of the present invention; and a pharmacologically acceptable carrier or excipient.

In one embodiment the present invention further relates to a method for modulating mesenchymal intercellular functions comprising the step of administering to a subject in need thereof a composition comprising a cDNA molecule according to the present invention. The polynucleotide sequences useful for the preparation of a pharmaceutical composition include polynucleotide sequences set forth in SEQ ID NOS: 9-17.

The polynucleotide molecules of the invention can be used to transfect human mesenchymal cells for inhibiting or suppressing proliferation. Thus the invention relates to compositions comprising said transfected human mesenchymal cells for use in disorders requiring inhibition or suppression of their intercellular interactions, such as in carcinomas.

In another embodiment the composition comprises human cells comprising a cDNA molecule according to the invention, in an amount effective to modulate their intercellular communication. Preferably, the cells are mesenchymal cells. In some embodiments the mesenchymal cells are autologous cells. The polynucleotide sequences useful for incorporation into a human cell are set forth in SEQ ID NOS: 9-17.

According to one currently preferred embodiment these methods are applicable in gene therapy.

In yet another aspect the present invention provides an antibody raised against at least one epitope of the truncated peptides or peptide derived from an intronic sequence of the present invention.

In one embodiment the molecules of the present invention are useful in the treatment of malignant diseases. The method can be carried out as an in vitro, ex vivo or in vivo procedure, especially in the form of gene therapy. According to one embodiment the method encompasses a method of treating a hyperproliferative disease in a subject in need thereof the method comprising the step of administering to the subject a therapeutically effective amount of an Ig heavy chain variant of the present invention. In some embodiments the Ig μ, heavy chain variant has an amino acid sequence set forth in any one of SEQ ID NOS: 1-3 or SEQ ID NOS: 7-8. In other embodiments the Ig δ heavy chain variant has an amino acid sequence set forth in any one of SEQ ID NOS: 4-6.

The invention further relates to a method for suppressing mesenchymal cell growth comprising the step of administering to a subject in need thereof a polynucleotide, a vector comprising polynucleotide or transfected mesenchymal and endothelial human cells comprising a polynucleotide molecule of the invention, in an amount effective to suppress cell proliferation. Preferably these transfected mesenchymal or endothelial cells will be autologous.

It will be appreciated by the skilled artisan that additional molecules may be involved in molecular complexes that regulate intercellular interactions together with the novel truncated variants of the present invention. It is also understood that the regulatory effect of the molecules of the invention may be either direct or indirect, the latter term expressing the need for additional molecular mediators or signals to achieve the observed biological effect.

According to the present invention mesenchymal Ig transcripts or antisense or RNAi thereto may be either directly or indirectly involved in the regulation of stem cell growth and differentiation. It is anticipated that additional molecular variants of the Ig superfamily will be transcribed in and expressed by mesenchymal and/or endothelial cells and these too are within the scope of the present invention. It will be appreciated by the skilled artisan that additional molecules may be involved in molecular complexes that regulate intercellular interactions together with the novel truncated variants of the present invention. It is also understood that the regulatory effect of the molecules of the invention may be either direct or indirect, the latter term expressing the need for additional molecular mediators or signals to achieve the observed biological effect.

In various embodiments, the method of the present invention is useful for promoting or inducing wound healing. In other embodiments the method is useful in suppressing cell proliferation, and can be used for example in cancer therapy.

These and other embodiments of the present invention will become apparent in conjunction with the figures, description and claims that follow.

### BRIEF DESCRIPTION OF THE DRAWINGS

FIGS. 1A-1D. Pre-BCR/BCR gene expression in mesenchyme: (A) RT-PCR analysis of cDNAs obtained from the MBA-2.1 cell line, WT MEF and IgM^{-/-} MEF. (1Ai) Expression of the constant regions of the different Ig isotypes; (1Aii) Expression of SLCs (surrogate light chain) and the pre-BCR accessory molecules. (1B) Northern blot analysis of Ig μHC transcripts: MBA-2.1 cells; IgM^{-/-} MEFs; and WT spleen. (1C) A scheme of RT-PCR analysis from three independent experiments. +: expression, −: no expression, +/−: inconsistent (some cell batches were positive). (1D) μHC expression by several murine mesenchymal cell lines and primary mesenchymal stem cells (MSCs).

FIG. 2. Early embryonic expression of un-rearranged transcripts of Ig μHC or Ig δHC: RT-PCR (Real time polymerase chain reaction) analysis using primers of Ig μHC or Ig δHC constant regions and for rearranged versions of these transcripts.

FIGS. 3A-3E. Increased incidence of defective morulae in IgM^{-/-} pregnancies and maternal origin of yolk sac IgM: Litter size and morulae properties: Litter size (3A) and total number of morulae (3B), and number of intact morulae (3C). (3D) (i) Western analysis using anti-IgM antibody. immunohistochemical staining using anti-IgM antibody of yolk sac from WT (ii) and IgM^{-/-} 12.5 dpc (days post coitus) embryos (iii). (3E) (i) Western blot analysis using anti-IgM antibody. Immunohistochemical analysis using anti-IgM antibody was performed on sections from 12.5 dpc WT embryo transplanted into IgM^{-/-} pseudo-pregnant recipient mother (ii) and 12.5 dpc IgM^{-/-} embryo transplanted into WT pseudo-pregnant recipient mother.

FIGS. 4A-4H. In situ hybridization localizes Ig μHC mRNA to embryonic mesenchyme: ³⁵S-labeled anti-sense RNA probe derived from the constant region of Ig μHC was used to hybridize WT (A-F) and IgM^{-/-} (4G, 4H) 12.5 dpc embryos. Transverse sections of WT (4A,4B) and IgM^{-/-} (4G,4H) embryos stained with Hematoxylin-Eosin (4A, 4C, 4D, 4E, 4F) and dark field views of image 4A (4B) and 4G (4H) are shown, as well as an enlargement of the boxed area in image 4A (4C). Arrows point to representative positive cells.

FIGS. 5A-5F. In situ hybridization detects Ig δHC RNA expressing cells in IgM^{-/-} embryos: ³⁵S-labeled anti-sense RNA probe derived from the constant region of Ig δHC was used to hybridize IgM^{-/-} (5A-5D) and WT (5E, 5F) 12.5 dpc embryo sections. Bright field image of 5A and 5E are shown in 5B and 5D respectively. 5C and 5D are enlarged images of areas in (5A) and the insets in these images show more details of the boxed areas. Arrows indicate positive cells.

FIGS. 6A-6D. Schematic structure of μ and δ HC mRNAs cloned from WT and IgM^{-/-} mouse embryonic fibroblasts respectively:

(6A) Schematic representation of exon-intron structure of the entire immunoglobulin heavy chain (HC) locus. (6B) The mesenchymal truncated Ig μHC mRNA transcripts: the two isoforms comprise six identical exons. The asterix (*) indicates the unique genomic sequence-TTCTAAAGGGGTCTATGATAGTGTGAC (SEQ ID NO: 18) found on this mRNA, (J2) JH2 sequence, (Cμ1-Cμ4) represents the Ig μHC constant region exons, (s) represents secreted form sequence (isoform i); and (m) represents the two exons of the transmembrane domain (isoform ii). (6C) A schematic enlargement of the δ constant HC region locus (1-7). (6D) Illustration of the mesenchymal truncated δHC transcripts: all four isoforms (i-iv) comprise the same first three exons; (Cδ1, CδH and Cδ3) represents the Ig δHC constant region exons (1-3), and the filled circle (·) indicates the unique genomic sequence-AAAGAATGGTATCAAAGGACAGTGCTTA GATCCAAGGTG (SEQ ID NO: 19)

FIGS. 7A-7D. Cμ mRNA encodes a 50 kDa protein that causes growth arrest upon overexpression: (7A) Cμ protein synthesis in a cell free system translation/transcription system using ³⁵S-methionine as the radiolabel for the newly synthesized protein (i) and detection of the protein by antibodies to IgM μchain, and protein expression of Cμ mRNA cloned in a mammalian expression vector and transfected into 293T cells (iii). (7B) Cellular localization of the cytosolic mesenchymal Cμ, or full-length Ig μHC. Immunofluorescence microscope analysis with anti-IgM antibodies was performed on cells transfected with cytosolic mesenchymal Cμ (i), or cytosolic full-length Ig μHC (ii) in 293T cells. (7C) Phase-contrast images of 293T cells 24 hours after transfection with empty vector (i); cytosolic mesenchymal Cμ (ii) and cytosolic full-length Ig μHC (iii). (7D) Overexpression of mesenchymal Cμ in 293T cells results in G1 arrest. (7Di) gating of cells stained positive and negative for IgM expression is shown in the middle panel. (7Dii) Cell cycle pattern of 293T cells overexpressing empty vector.

### DETAILED DESCRIPTION OF THE INVENTION

The present invention discloses isolated novel polypeptide and peptide variants of members of the Immunoglobulin superfamily, in cells that do not belong to hemopoietic or lymphoid lineages. Hitherto these molecules were considered to be specific to lymphoid lineages, with the exception of certain transformed cell lines or tumors that were known to express certain abnormal transcripts of these genes. Importantly, the novel transcripts now discovered in mesenchymal cells are also translated and expressed as novel truncated variants of Ig molecules by these cells.

These novel truncated variants are capable of regulating cell growth and differentiation, as well as mediating cell-cell interactions. These attributes can be used to stimulate cell growth, for instance especially in order to enhance hemopoiesis. These methods should prove particularly useful in situations involving bone marrow transplantation, by way of example. In contradistinction, these attributes can be used to suppress cell growth, for instance in order to prevent cancer growth or metastasis. The growth stimulation might entail gene therapy, while the growth suppression might entail either antisense therapy or antibody targeting or other methods known in the art.

The present invention resulted in part from studies on the interactions of stromal cell lines with thymic T cells, during which reverse transcription polymerase chain reaction (RT-PCR) was used to amplify TCR gene fragments. Unexpectedly, the MBA-13 mesenchymal stromal cell line, derived from mouse bone marrow, was found to consistently express TCRβ constant (Cβ) region, while cDNA from a negative control tissue, i.e. liver, and from several control cell lines such as pre-B cells, plasmacytoma and mastocytoma cells, did not produce PCR products using primers from the TCR gene.

Further studies with a variety of stromal cell lines, showed the existence of TCR gene derived mRNAs that encode short versions of the gene consisting of the constant (C) domain, which is identical to that of T cell receptor, a joining (J) region, which may be one of several alternatives, and a 5′ sequence corresponding to an intronic J sequence (again one of several alternatives) including an in-frame codon for methionine (see Barda-Saad et al 2002). This mRNA lacks V region sequences. One of such molecules, namely a new version of a TCRβ2.6, was shown to exist in mesenchymal cells and to encode a cell surface mesenchymal protein. Expression on the mRNA level has also been observed in the thymus (see Barda-Saad, 2002).

The finding that mesenchymal cells express TCR genes raised the possibility that other members of the immunoglobulin (Ig) superfamily are expressed in the mesenchyme. A series of stromal mesenchymal cell lines derived in our laboratory including one subtype that shares properties with endothelial cells (MBA-2.1 cells) were screened. Based on our experience with truncated TCR molecules, which were found to be lacking the variable part and possessing a J region preceded by an intronic sequence including a codon for methionine, a PCR analysis on MBA-2.1 cells and found that they do express mRNA transcripts corresponding to truncated Ig μ heavy chains. Therefore at least one type, and possibly more, of stromal cells express the Ig μ chains and may present this protein as a surface molecule.

The ability of the truncated immunoglobulin superfamily variants expressed in mesenchymal cells to regulate or modulate growth/differentiation control of their neighboring cells are further disclosed. In other words, not only do the novel molecules of the invention modulate the growth of the mesenchymal cells themselves but they are also capable of regulating the growth and differentiation of hemopoietic stem cells. Moreover, they are capable of regulating the growth of transformed cells.

The present invention discloses the novel uses of truncated Ig variants.

The Ig chain, are now disclosed herein to be linked to the cell-cell interactions, cell growth and differentiation and thus can be used to control stromal functions. The TCR appears to be most abundant in mesenchymal stroma whereas the μ chain originally thought to be abundant in endothelial stroma is now shown to be expressed in mesenchymal stem cells.

It is anticipated that additional molecular variants of the Ig superfamily will be transcribed and expressed on mesenchymal and/or endothelial cells and these too are within the scope of the present invention. It will be appreciated by the skilled artisan that additional molecules may be involved in molecular complexes that regulate intercellular interactions together with the novel truncated variants of the present invention.

Definitions

For convenience and clarity certain terms employed in the specification, examples and claims are described herein.

“Nucleic acid sequence” or “polynucleotide” as used herein refers to an oligonucleotide or nucleotide and fragments or portions thereof, and to DNA or RNA of genomic or synthetic origin, which may be single- or double-stranded, and represent the sense or antisense strand. cDNA refers to complementary DNA, a single-stranded DNA that is complementary to mRNA transcript.

Similarly, “amino acid sequence” as used herein refers to an oligopeptide, peptide, polypeptide, or protein sequence, and fragments or portions thereof, and to naturally occurring, synthetic or recombinant molecules. The terms listed herein are not meant to limit the amino acid sequence to the complete, wild type amino acid sequence associated with the recited protein molecule. Natural coded amino acids and their derivatives are represented by either the one-letter code or three-letter codes according to IUPAC conventions. When there is no indication, the L isomer is used.

The term “variant” as used herein refers to a polypeptide sequence that possesses some modified structural property of the wild type or parent protein. For example, the variant may be truncated at either the amino or carboxy terminus or both termini or may have one or more amino acids deleted, inserted and or substituted.

A “polynucleotide” as used herein refers to DNA or RNA of genomic or synthetic origin, having more than about 100 nucleic acids.

The term “RNAi molecule” or “RNAi oligonucleotide” refers to single- or double-stranded RNA molecules having a total of about 15 to about 100 bases, preferably from about 30 to about 60 bases and comprises both a sense and antisense sequence. For example the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

“Complementarity” refers to the ability of a nucleic acid to form hydrogen bond(s) with another nucleic acid sequence. A percent complementarity indicates the percentage of contiguous residues in a nucleic acid molecule which can form hydrogen bonds (e.g., Watson-Crick base pairing) with a second nucleic acid sequence. “Fully complementary” means that all the contiguous residues of a nucleic acid sequence will hydrogen bond with the same number of contiguous residues in a second nucleic acid sequence. The term “substantially” complementary as used herein refers to a molecule in which about 80% of the contiguous residues of a nucleic acid sequence will hydrogen bond with the same number of contiguous residues in a second nucleic acid sequence. In some embodiments substantially complementary refers to 85%, 90%, 95% of the contiguous residues of a nucleic acid sequence hydrogen bonding with the same number of contiguous residues in a second nucleic acid sequence.

The ribo-oligonucleotide strands according to the present invention each comprise from about 12 to about 100 nucleotides, preferably from about 12 to about 50 nucleotides. In some embodiments the ribo-oligonucleotides of the present invention each comprise from about 17 to about 28 nucleotides. In other embodiments each ribo-oligonucleotide strand comprises about 19 to about 21 oligonucleotides. The ribo-oligonucleotides according to the invention can be produced synthetically or by recombinant techniques.

The term “expression vector” and “recombinant expression vector” as used herein refers to a DNA molecule, for example a plasmid or virus, containing a desired and appropriate nucleic acid sequences necessary for the expression of the operably linked RNAi sequence for expression in a particular host cell. A suitable example includes a plasmid with a sequence encoding a small hairpin RNA (shRNA) under the control of an RNA Polymerase III (Pol III) promoter. A particularly suitable vector directs expression of a truncated Ig antisense or RNAi molecule when introduced into a cell, thereby reduce the levels of endogenous Ig expression.

As used herein “operably linked” refers to a functional linkage of at least two sequences. Operably linked includes linkage between a promoter and a second sequence, for example an oligonucleotide of the present invention, wherein the promoter sequence initiates and mediates transcription of the DNA sequence corresponding to the second sequence.

The term “expression product” is used herein to denote a truncated Ig polypeptide, or an antisense or RNAi oligonucleotide. An Ig RNAi expression product is preferably siRNA or shRNA.

A “subject” refers to a mammalian recipient or host of the composition of the present invention. In some embodiments the subject is a human subject.

The Endothelium

The cellular and molecular mechanisms that allow for the maintenance of hemopoietic stem cells are inadequately understood. Morphological examination of various embryonic hemopoietic sites revealed that hemopoietic progenitor cells are in close physical contact with the endothelium in both yolk sac and aorta-gonado-mesonephros region (AGM) (Lin et al 1995). The close association in the development of hemopoietic and endothelial cells during embryonic life (Garcia Porrero et al. 1995) has led to the hypothesis that the two lineages may derive from a common precursor called hemangioblast. Recently several authors reported that endothelial cells, both vascular endothelial cells and bone marrow endothelial cells, support hemopoiesis (Bagdy & Heinrich 1991). The mechanism by which the endothelial cells support hemopoiesis is thought to involve endothelial cell derived cytokines (Fleischman et al 1995), extracellular matrix proteins (Rafii et al 1994) and cell-cell interactions (Fina et al 1994). Stromal cells are thought to be an essential component of the lymphohematopoietic microenvironment. B lymphocytes develop in the liver during fetal life and in the bone marrow of adult animals (Kincade et al 1981). It has been suspected that yet unknown stromal cell molecules may be involved in B-lineage cell growth and development (Palacios and Samaridis 1992).

Mesenchymal Cells

Mesenchymal cells play a central role in embryogenesis by directing organogenesis. In the adult organism, tissue remodeling, such as that occurring in wound healing, is initiated by mesenchymal fibroblasts. The study of regulation of hemopoiesis demonstrated that blood cell formation is locally regulated by stromal mesenchyme (Zipori, 1989; Zipori et al., 1989; Zipori, 1990; Weintroub et al., 1996). Indeed, bone marrow-derived primary stroma as well as a variety of mesenchymal cells lines derived from primary bone marrow cultures exhibit an in vitro capacity to support hemopoiesis and, upon transplantation, promote the formation of bone and hemopoietically active tissue in vivo at the site of transplantation. The molecules that mediate the instructive stromal activities have been shown to be a variety of cytokines and adhesion molecules. However, the molecules identified thus far cannot account for the wide spectrum of stromal cell functions and certainly do not explain stroma organization, stem cell renewal and other vital stromal functions.

Mesenchymal cells from the bone marrow are well known to be obligatory for the maintenance and renewal of hemopoietic stem cells in vitro, and these cells are critical for the maintenance of hemopoiesis in vivo. This function of the mesenchyme is not restricted to blood cells. In fact, every tissue and organ is composed of a stromal mesenchyme support that interacts with the other, tissue specific cell types. Thus, the growth and differentiation of cells within different tissues, and the development of tumors, are all dependent on mesenchymal functions.

Knockout Mice

Loss-of-function experiments in mice are mostly done by the technique of gene knockout. Knock-out mice employed in the present invention demonstrate the important role played by immunoglobulin superfamily variants in hemopoiesis as exemplified herein below. The technology is well known in the art. It requires the use of mouse genes for the purpose of generating knockout of the specific gene in embryonic stem (ES) cells that are then incorporated into the mouse germ-line cells from which mice carrying the gene knockout are generated. From a human gene there are several ways to recover the homologous mouse gene. One way is to use the human gene to probe mouse genomic libraries of lambda phages, cosmids or BACs. Positive clones are examined and sequenced to verify the identity of the mouse gene. Another way is to mine the mouse EST database to find the matching mouse sequences. This can be the basis for generating primer-pairs or specific mouse probes that allow an efficient screen of the mouse genomic libraries mentioned above by PCR or by hybridization. For the vast majority of genes the mouse homologue of the human gene retains the same biological function. The loss-of-function experiments in mice indicate the consequences of absence of expression of the gene on the phenotype of the mouse and the information obtained is applicable to the function of the gene in humans. On many occasions a specific phenotype observed in knockout mice was similar to a specific human inherited disease and the gene then proved to be involved and mutated in the human disease.

Introduction of Proteins Peptides, and DNA into Cells

The present invention provides proteins encoded by the truncated immunoglobulin superfamily variant genes, peptides derived therefrom and antisense DNA molecules based on the variant gene transcripts. A therapeutic or research-associated use of these tools necessitates their introduction into cells of a living organism or into cultured cells. For this purpose, it is desired to improve membrane permeability of peptides, proteins and antisense molecules. The same principle, namely, derivatization with lipophilic structures, may also be used in creating peptides and proteins with enhanced membrane permeability. For instance, the sequence of a known membranotropic peptide may be added to the sequence of the peptide or protein. Further, the peptide or protein may be derivatized by partly lipophilic structures such as the above-noted hydrocarbon chains, which are substituted with at least one polar or charged group. For example, lauroyl derivatives of peptides have been described in the art. Further modifications of peptides and proteins include the oxidation of methionine residues to thereby create sulfoxide groups and derivatives wherein the relatively hydrophobic peptide bond is replaced by its ketomethylene isoester (COCH₂) have been described. It is known to those of skill in the art of protein and peptide chemistry these and other modifications enhance membrane permeability.

Another way of enhancing membrane permeability is to make use of receptors, such as virus receptors, on cell surfaces in order to induce cellular uptake of the peptide or protein. This mechanism is used frequently by viruses, which bind specifically to certain cell surface molecules. Upon binding, the cell takes the virus up into its interior. The cell surface molecule is called a virus receptor. For instance, the integrin molecules CAR and AdV have been described as virus receptors for Adenovirus. The CD4, GPR1, GPR15, and STRL33 molecules have been identified as receptors/coreceptors for HIV.

By conjugating peptides, proteins or oligonucleotides to molecules that are known to bind to cell surface receptors, the membrane permeability of said peptides, proteins or oligonucleotides will be enhanced. Examples of suitable groups for forming conjugates are sugars, vitamins, hormones, cytokines, transferrin, asialoglycoprotein, and the like molecules. U.S. Pat. No. 5,108,921 describes the use of these molecules for the purpose of enhancing membrane permeability of peptides, proteins and oligonucleotides, and the preparation of said conjugates. Folate or biotin may be used to target the conjugate to a multitude of cells in an organism, because of the abundant and nonspecific expression of the receptors for these molecules.

The above use of cell surface proteins for enhancing membrane permeability of a peptide, protein or oligonucleotide of the invention may also be used in targeting the peptide, protein or oligonucleotide of the present invention to certain cell types or tissues. For instance, if it is desired to target neural cells, it is preferable to use a cell surface protein that is expressed more abundantly on the surface of those cells.

The protein, peptide or polynucleotide of the invention may therefore, using the above-described conjugation techniques, be targeted to mesenchymal cells. For instance, if it is desired to enhance mesenchymal cell growth in order to augment autologous or allogeneic bone marrow transplantation or wound healing, then the immunoglobulin superfamily variant genes could be inserted into mesenchymal cells as a form of gene therapy. In this embodiment, local application of the cells containing the cDNA molecule can be used to modulate mesenchymal cell-cell interactions with neighboring cells in the microenvironment thus enhancing the wound healing process

In contrast, it is often desirable to inhibit mesenchymal cell-cell interactions, as in the case of a tumor. Therefore, mesenchymal cells of the tumor can be transfected with the antisense cDNA and then be used for treatment of localized solid tumors, to achieve regression of the tumor by blocking mesenchyme intercellular communication.

The proteins encoded by the mRNAs of the invention are cell surface receptors of mesenchymal cells and may probably interact with ligands presented by neighboring hemopoietic or non-hemopoietic cells. Thus, in bound or soluble form, these proteins or the peptides derived therefrom, may have modulatory effects on cells that bear said ligands.

Antibodies

The present invention also comprehends antibodies specific for the polypeptides or peptides encoded by the truncated immunoglobulin superfamily variant transcripts, which are part of the present invention as discussed above. The proteins and peptides of the invention may be used as immunogens for production of antibodies that may be used as markers of mesenchymal cells. Such an antibody may be used for diagnostic purposes to identify the presence of any such naturally-occurring proteins. Such antibody may be a polyclonal antibody or a monoclonal antibody or any other molecule that incorporates the antigen-binding portion of a monoclonal antibody specific for such a protein. Such other molecules may be a single-chain antibody, a humanized antibody, a F(ab) or F(ab′)₂ fragment, a chimeric antibody, an antibody to which is attached a label, such as fluorescent or radioactive label, or an immunotoxin in which a toxic molecule is bound to the antigen binding portion of the antibody. The examples are intended to be non-limiting. However, as long as such a molecule includes the antigen-binding portion of the antibody, it will be expected to bind to the protein and, thus, can be used for the same diagnostic purposes for which a monoclonal antibody can be used.

In some embodiments the antibody is an antibody against a polypeptide sequence encoded by the intronic sequence, or to a fragment thereof. In other embodiments the antibody is directed to a C region or fragment thereof.

Polynucleotide Sequences

The present invention also provides for an isolated nucleic acid molecule, which comprises a polynucleotide sequence encoding the polypeptide of the invention and a host cell comprising this nucleic acid molecule. Furthermore, also within the scope of the present invention is a nucleic acid molecule containing a polynucleotide sequence having at least 90% sequence identity, preferably about 95%, and more preferably about 97% identity to the above encoding nucleotide sequence as would well understood by those of skill in the art.

The invention also provides isolated nucleic acid molecules that hybridize under high stringency conditions to polynucleotides having SEQ ID NO: 9 through SEQ ID NO: 17 or the complement thereof. As used herein, highly stringent conditions are those which are tolerant of up to about 5-20% sequence divergence, preferably about 5-10%. Without limitation, examples of highly stringent (−10° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the wash conditions, particularly if the hybridization conditions used are those which allow less stable hybrids to form along with stable hybrids. The wash conditions at higher stringency remove the less stable hybrids. A common hybridization condition that can be used with the highly stringent to moderately stringent wash conditions described above may be performed by hybridizing in a solution of 6×SSC (or 6×SSPE), 5×Denhardt's reagent, 0.5% SDS, 100 μg/ml denatured, fragmented salmon sperm DNA at an appropriate incubation temperature Ti. (See generally Sambrook et al., Molecular Cloning: A Laboratory Manual, 2d edition, Cold Spring Harbor Press (1989)) for suitable high stringency conditions.

Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization and hybridization duration. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching. Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al (1984), as
Tm=81.5° C.+16.6(log M)+0.41(% GC)−0.61(% form)−500/L
and for DNA:RNA hybrids, as
Tm=79.8° C.+18.5(log M)+0.58(% GC)−11.8(% GC)²−0.56(% form)−820/L
where M, molarity of monovalent cations, 0.01-0.4 M NaCl,
% GC, percentage of G and C nucleotides in DNA, 30%-75%,
% form, percentage formamide in hybridization solution, and
L, length hybrid in base pairs.

Tm is reduced by 0.5-1.5° C. (an average of 1° C. can be used for ease of calculation) for each 1% mismatching. The Tm may also be determined experimentally.

Filter hybridization is typically carried out at 68° C. , and at high ionic strength (e.g., 5-6×SSC), which is non-stringent, and followed by one or more washes of increasing stringency, the last one being of the ultimately desired high stringency. The equations for Tm can be used to estimate the appropriate Ti for the final wash, or the Tm of the perfect duplex can be determined experimentally and Ti then adjusted accordingly.

The present invention also relates to a vector comprising the nucleic acid molecule of the present invention. The vector of the present invention may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

Antisense or RNAi Sequence

As will be exemplified herein below, the expression or lack of expression of the immunoglobulin heavy chains seems to control interactions of the mesenchyme with other neighboring cells, especially in the process of hemopoiesis. The invention therefore further relates to the use of the cDNA, antisense and RNAi molecules of the invention derived from Ig HC mRNAs for expression in cells and tissues for the purpose of modulating stromal/mesenchymal interactions and cell-cell communication with their neighbors in the microenvironment of the tissue involved.

For this purpose, the cDNA antisense or RNAi molecule is inserted in appropriate vectors such as, but not limited to, the retroviral vectors DCAl and DCMm that have been used in clinical trials in gene therapy (Bordignon et al., 1995). Preferably, the vector containing the molecule, under the control of a suitable promoter such as that cDNA's own promoter, will be used to infect or transfect suitable mammalian, preferably human, most preferably the patient's autologous mesenchymal cells. The genetically-modified mesenchymal cells are then administered to a patient in need thereof by an appropriate route and are expressed in the desired site or tissue.

In order to manipulate the expression of an undesirable gene, it is necessary to produce antisense RNA or RNAi in a cell. To this end, the complete or partial cDNA of an undesirable gene in accordance with the present invention is inserted into an expression vector comprising a promoter. The 3′ end of the cDNA is thereby inserted adjacent to the 3′ end of the promoter, with the 5′ end of the cDNA being separated from the 3′ end of the promoter by said cDNA. Upon expression of the cDNA in a cell, an antisense RNA is therefore produced which is incapable of coding for the protein. The presence of antisense RNA in the cell reduces the expression of the cellular (genomic) copy of the undesirable gene.

For the production of antisense RNA, the complete cDNA may be used. Alternatively, a fragment thereof may be used, which is preferably between about 9 and 2,000 nucleotides in length, more preferably between 15 and 500 nucleotides, and most preferably between 30 and 150 nucleotides.

Any sequence may be selected as the target sequence for antisense inhibition yet, the target sequence preferably corresponds to a region within the 5′ half of the cDNA, more preferably the 5′ region comprising the 5′ untranslated region and/or the first exon region, and most preferably comprising the ATG translation start site. Alternatively, the fragment may correspond to DNA sequence of the 5′ untranslated region only.

A synthetic oligonucleotide may be used as antisense oligonucleotide. The oligonucleotide is preferably a DNA oligonucleotide. The length of the antisense oligonucleotide is preferably between 9 and 150, more preferably between 12 and 60, and most preferably between 15 and 50 nucleotides. Suitable antisense oligonucleotides that inhibit the production of the protein of the present invention from its encoding mRNA can be readily determined with only routine experimentation through the use of a series of overlapping oligonucleotides similar to a “gene walking” technique that is well-known in the art. Such a “walking” technique as well-known in the art of antisense development can be done with synthetic oligonucleotides to walk along the entire length of the sequence complementary to the mRNA in segments on the order of 9 to 150 nucleotides in length. This “gene walking” technique will identify the oligonucleotides that are complementary to accessible regions on the target mRNA and exert inhibitory antisense activity.

Alternatively, an oligonucleotide based on the coding sequence of a protein capable of binding to an undesirable gene or the protein encoded thereby can be designed using known algorithms, for example Oligo 4.0 (National Biosciences, Inc.). Antisense molecules may also be designed to inhibit translation of an mRNA into a polypeptide by preparing an antisense which will bind in the region spanning approximately −10 to +10 nucleotides at the 5′ end of the coding sequence.

Modifications of oligonucleotides that enhance desired properties are generally used when designing antisense oligonucleotides. For instance, phosphorothioate bonds are used instead of the phosphoester bonds that naturally occur in DNA, mainly because such phosphorothioate oligonucleotides are less prone to degradation by cellular enzymes. Preferably, a 2′-methoxyribonucleotide modification in 60% of the oligonucleotides is used. Such modified oligonucleotides are capable of eliciting an antisense effect comparable to the effect observed with phosphorothioate oligonucleotides.

Therefore, the preferred antisense oligonucleotide of the present invention has a mixed phosphodiester-phosphorothioate backbone. Preferably, 2′-methoxyribonucleotide modifications in about 30% to 80%, more preferably about 60%, of the oligonucleotide are used.

In the practice of the invention, antisense oligonucleotides or antisense RNA may be used. The length of the antisense RNA is preferably from about 9 to about 3,000 nucleotides, more preferably from about 20 to about 1,000 nucleotides, most preferably from about 50 to about 500 nucleotides.

In order to be effective, the antisense oligonucleotides of the present invention must travel across cell membranes. In general, antisense oligonucleotides have the ability to cross cell membranes, apparently by uptake via specific receptors. As the antisense oligonucleotides are single-stranded molecules, they are to a degree hydrophobic, which enhances passive diffusion through membranes. Modifications may be introduced to an antisense oligonucleotide to improve its ability to cross membranes. For instance, the oligonucleotide molecule may be linked to a group, which includes a partially unsaturated aliphatic hydrocarbon chain, and one or more polar or charged groups such as carboxylic acid groups, ester groups, and alcohol groups. Alternatively, oligonucleotides may be linked to peptide structures, which are preferably membranotropic peptides. Such modified oligonucleotides penetrate membranes more easily, which is critical for their function and may, therefore, significantly enhance their activity.

Gene Therapy

On the other hand it may be important to increase the expression of the truncated Ig HC gene in conditions requiring modified intercellular mesenchymal interactions such as in improper wound healing, or in tumor therapy, for example by means of gene therapy. It was shown that TCR affects hemopoiesis, and it is likely that the Ig heavy chain variants have similar or complementary functions.

Recently, gene transfer into hematopoietic cells using viral vectors has focused mostly on lymphocytes and hematopoietic stem cells (HSCs). HSCs have been considered particularly important as target cells because of their pluripotency and ability to reconstitute hemopoiesis after myeloablation and transplantation. HSCs are believed to have the ability to live a long time, perhaps a lifetime, in the recipient following bone marrow transplantation. Genetic correction of HSCs can therefore potentially last a lifetime and permanently cure hematologic disorders in which genetic deficiencies cause the pathology. Oncoretroviral vectors have been the main vectors used for HSCs because of their ability to integrate into the chromosomes of their target cells. Gene-transfer efficiency of murine HSCs is high using oncoretroviral vectors. In contrast, gene-transfer efficiency using the same viral vectors to transduce human HSCs or HSCs from large animals has been much lower. Although these difficulties may have several causes, the main reason for the low efficiency of human HSC transduction with oncoretroviral vectors is probably because of the nondividing nature of HSCs. Murine HSCs can be easily stimulated to divide in culture, whereas it is more problematic to stimulate human HSCs to divide rapidly in vitro. Because oncoretroviral vectors require dividing target cells for successful nuclear import of the preintegration complex and subsequent integration of the provirus, only the dividing fraction of the target cells can be transduced.

In addition, adenovirus (Adv)-mediated gene transfer has recently gained new attention as a means to deliver genes for hematopoietic stem cell (HSC) or progenitor cell gene therapy. In the past, HSCs have been regarded as poor Adv targets, mainly because they lack the specific Adv receptors required for efficient and productive Adv infection. In addition, the nonintegrating nature of Adv has prevented its application to HSC and bone marrow transduction protocols where long-term expression is required. There is even controversy as to whether Adv can infect hematopoietic cells at all. In fact, the ability of Adv to infect epithelium-based targets and its inability to effectively transfect HSCs have been used in the development of eradication schemes that use Adv to preferentially infect and “purge” tumor cell-contaminating HSC grafts. However, there are data supporting the existence of productive Adv infections into HSCs. Such protocols involve the application of cytokine mixtures, high multiplicities of infection, long incubation periods, and more recently, immunological and genetic modifications to Adv itself to enable it to efficiently transfer genes into HSCs. This is a rapidly growing field, both in terms of techniques and applications.

Pharmaceutical Compositions

The present invention also provides for a composition comprising at least one polypeptide or polynucleotide of the present invention. “Therapeutic” refers to any pharmaceutical, drug or prophylactic agent which may be used in the treatment (including the prevention, diagnosis, alleviation, or cure) of a malady, affliction, disease or injury in a patient. Therapeutically useful peptides, polypeptides and polynucleotides may be included within the meaning of the term pharmaceutical or drug.

The term “excipient” or “carrier” as used herein refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols. Pharmaceutical compositions may also include one or more additional active ingredients.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

The pharmaceutical composition of this invention may be administered by any suitable means, such as orally, intranasally, subcutaneously, intramuscularly, intravenously, intra-arterially, or parenterally. Ordinarily, intravenous (i.v.) or parenteral administration will be preferred.

The pharmaceutical composition of the invention generally comprises a buffering agent, an agent which adjusts the osmolarity thereof, and optionally, one or more carriers, excipients and/or additives as known in the art, e.g., for the purposes of adding flavors, colors, lubrication, or the like to the pharmaceutical composition.

Carriers are well known in the art and may include starch and derivatives thereof, cellulose and derivatives thereof, e.g., microcrystalline cellulose, xanthan gum, and the like. Lubricants may include hydrogenated castor oil and the like.

A preferred buffering agent is phosphate-buffered saline solution (PBS), which solution is also adjusted for osmolarity.

A preferred pharmaceutical formulation is one lacking a carrier. Such formulations are preferably used for administration by injection, including intravenous injection.

The preparation of pharmaceutical compositions is well known in the art and has been described in many articles and textbooks.

Additives may also be selected to enhance uptake of the polynucleotides or antisense oligonucleotide across cell membranes. Such agents are generally agents that will enhance cellular uptake of double-stranded DNA molecules. For instance, certain lipid molecules have been developed for this purpose, including the transfection reagents DOTAP (Boehringer Mannheim), Lipofectin®, Lipofectam® and Transfectam®, which are available commercially. The antisense or RNAi oligonucleotide of the invention may also be enclosed within liposomes.

The preparation and use of liposomes, e.g., using the above-mentioned transfection reagents, is well known in the art. Other methods of obtaining liposomes include the use of Sendai virus or of other viruses.

The above-mentioned cationic or nonionic lipid agents not only serve to enhance uptake of oligonucleotides into cells, but also improve the stability of oligonucleotides that have been taken up by the cell.

Methods of Use

As used herein the terms “treating” or “treatment” should be interpreted in their broadest possible context. Accordingly, “treatment” broadly includes amelioration of the symptoms or severity of a particular disorder, for example a reduction in the rate of cell proliferation, reduction in the growth rate of a tumor, partial or full regression of a tumor, or preventing or otherwise reducing the risk of metastases or of developing further tumors. Treatment may also refer to the healing or repair of tissue, for example wound healing.

As used herein, a “therapeutically effective amount”, or an “effective amount” is an amount necessary to at least partly attain a desired response.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

Materials and Methods

Isolation and Transfer of Embryonic Cells

Balb/c and IgM-deficient (on Balb/c background) (Lutz, 1998), were maintained under pathogen-free conditions, crossed, and homozygous IgM^{-/-} as well as IgM^{+/+} (WT) mice were selected. Superovulation was induced in virgin 5 week old mice by intraperitoneal injection of 5 units PMSG (Sigma Chemical, St Louis, Mo.) and 5 units hCG (Sigma) 48 hours later. The following day, females were killed and unfertilized oocytes were collected from the oviduct by flushing M2 medium containing hyaluronidase (300 mg/ml). To isolate morulae, superovulation was induced as above in 4-6 week old virgin Balb/c, ICR and IgM^{-/-} mice. Each superovulated mouse was then placed in a cage overnight with a sexually mature male of the same strain. Successful mating was determined by the presence of a copulation plug on the following day (designated as day 0.5 of gestation). Females were killed on day 2.5 and morulae were collected by flushing M2 medium through the uteri. Embryos cultured in vitro were placed into 30 μl drops of M2 medium with 4 mg/ml BSA and covered with light paraffin oil. Embryos were sorted into their respective developmental stage and defective embryos were microscopically identified. For embryo transfer, recipient female mice were prepared by mating with vasectomized males (ICR) 2.5 days before the embryo transfer. The procedure of embryo transfer was performed by implanting morulae into pseudo-pregnant recipient females. Fifteen morulae were transferred to each uterine horn (total 30 per female). The mice were killed 10 days following the embryo transfer. MEF were derived from 12.5-day-old embryos.

MSC Production

BM cells were obtained from 7-8 week old female C57BL/6 mice. MSC were grown in murine Mesencult™ basal Media supplemented with 20% murine mesenchymal supplement (StemCell Technologies Va, CA) containing 60 μg/ml penicillin, 100 μg/ml streptomycin and incubated at 37° C. in a humidified incubator with 10% CO₂ in air. Half of the medium was replaced every 3 days to remove the non-adherent cells. Once the adherent cells had reached confluence, the cells were trypsinized, centrifuged and re-suspended in their medium and incubated with antibodies specific to CD45.2 R-phycoerythrin (RPE) (Southern Biotechnology Associates, Birmingham, Ala.) and CD11b/Mac1 fluorescein isothiocyanate (FITC) (Southern Biotechnology Associates, Birmingham, Ala.), for 1 hour. The cells were sorted using FACSVANTAGE cell sorter (FACSVANTAGE SE, Becton Dickinson Immunocytometry System, San Jose Calif.). The double negative cell population was collected and seeded in MSC medium.

Cell Lines and Transfection Procedure

Murine bone marrow-derived stromal cell lines MBA-2.1 and MBA-2.4 endothelial-like, MBA-13 fibroendothelial, MBA-15 osteogenic and 14F1.1 preadipocytes (Zipori, 1989; Zipori, 1985) and the 293T human embryonic kidney cell line were used. These were cultured in DMEM supplemented with 100 μM glutamine and 10% FCS, and containing 60 μg/ml penicillin, 100 μg/ml streptomycin and 50 mg/L kanamycin and incubated at 37° C. in a humidified incubator with 10% CO₂ in air. Transient DNA transfections were done as follows: 1.5×10⁵ 293T cells were plated in each well of a 6-well plate (Corning) a day previous to transfection. Plasmid DNA (1.5 μg) was transfected to 293T cells by the calcium-phosphate/DNA precipitation method.

Flow Cytometry and Immunohistochemistry

293T cells (1×10⁶/100-mm-diameter dish) were transfected and fixed in 100% methanol for 30 minutes, collected by low-speed centrifugation and re-suspended in PBS, incubated for 40 minutes with primary antibody anti-IgM (A90-101A, 1:700; Bethyl Laboratories, Montgomery, Tex.) for 45 minutes followed by 40 minutes incubation with biotin-conjugated donkey anti-goat antibody (AP180B, 1:1500; Chemicon, Temecula, Calif.) and finally by 40 minutes staining with Oregon Green® 488-conjugated streptavidin (Molecular Probes Eugene, Oreg.). Cells were re-suspended in PBS containing 50 μg/ml RNAse A (Sigma) and 50 μg/ml propidium iodide (Sigma), and incubated in the dark at 37° C. for 30 minutes and 10,000-20,000 cells were analyzed for DNA content by FACScan (Becton Dickinson, San Jose, Calif.). Histograms were prepared using CellQuest™ software. For immunohistochemistry embryos were fixed in 4% (vol/vol) phosphate-buffered formalin, dehydrated, embedded in paraffin, sections were prepared, boiled for 10 minutes in 10 mM citrate buffer pH 6.0 and cooled down for at least 2 hours. Sections were then blocked and permeabilized for 30 minutes at room temperature using a blocking solution (10% normal horse serum (NHS), and 0.1% Triton X-100, in PBS), incubated overnight at room temperature with biotinylated goat anti-mouse IgM antibody (115-065-075, 1:2000, Jackson) and then with peroxidase-labeled avidin-biotin complex (ABC-complex; K-0377, DAKO, Glostrup, DK). Sections were then washed and developed in diamino-benzidine (DAB) reagent (Sigma), rinsed in water, counter-stained with hematoxylin, mounted in Enthellan (Merck) and were analyzed using light microscope (Nikon Eclipse E800).

Immunofluorescence

Human embryonic kidney (HEK) 293T cells (1.5×10⁵) were seeded on glass cover slips (13 mm in diameter). Twenty four hours after transfection cells were fixed, permeabilized and incubated with goat anti-IgM (A90-101A, 1:700; Bethyl) for 45 minutes, washed in PBS, incubated 40 minutes with biotin-conjugated donkey anti-goat antibody (AP180B, 1:1500; Chemicon) and then stained 40 minutes with Oregon Greene® 488-conjugated streptavidin (Molecular Probes) and washed in PBS. Cells were viewed and photographed using Nikon E 1000 and the Openlab 4.0.1 software.

Plasmid Construction

The expression constructs of cytosolic and transmembrane mesenchymal Ig μHC was generated as follows: transcripts were cloned from the MBA-2.1 cDNA library into pCANmycA vector (Stratagene, La Jolla, Calif.). The cytosolic mesenchymal Ig μHC was amplified using the sense primer

| | |
|---|---|
| 5′-CCGGAATTCGGCTGCCTAGCCCGGGACTTC | (SEQ ID NO: 20) |
| C-3′ | |
| and the antisense primer | |
| 5′-CGGCTCGAGTCAATAGCAGGTGCCGCCTGT | (SEQ ID NO: 21) |
| GTC-3′, | |
| the transmembrane mesenchymal Ig | |
| μHC was amplified using the sense | |
| primer | |
| 5′-CCGGAATTCGGCTGCCTAGCCCGGGACTTC | (SEQ ID NO: 22) |
| C-3′ | |
| and the antisense primer | |
| 5′-CGGCTCGAGTCATTTCACCTTGAACAGGGT | (SEQ ID NO: 23) |
| GACG-3′ | |

both fragments were digested with XhoI and EcoRI and ligated into pCANmycA vector. Another construct of the cytosolic mesenchymal Ig μHC was designed for the cell-free transcription/translation assay described. The insert was cloned into the vector pBluescript II KS (+/−) (Stratagene) using the same primers (only the XhoI restriction site was modified by NtoI). The vector containing the cytosolic form of the full-length Ig μHC was a kind gift from Dr. Yair Argon (University of Chicago).
Cell-free Transcription/Translation

The transcription/translation experiment was performed by means of the TNT quick-coupled transcription/translation system (Promega, Madison, Wis.) according to the instructions of the producer.

Northern Blots

Total RNA was extracted using TriReagent (Molecular Research Center, Cincinnati) and 2-40 μg samples were hybridized with the μHC constant region probe. Separation of RNA samples by electrophoresis was performed on 1% agarose, 5.2% formaldehyde (37% solution), 1×MOPS gels. RNA was transferred to a Hybondg®-N membrane (Amersham). The blot was hybridized at 68° C. for 60 minutes in express hybridization solution containing [α-³²P] labeled probe. After washing the blot was exposed to X-ray film (Kodak).

Western Blots

Cells were harvested in 400 μl ice-cold RIPA lysis buffer (50 mM Tris, 150 mM NaCl, 1% nonidet P-40, 0.5% deoxycholate, 0.1% SDS, 10% glycerol and 1 mM EDTA pH 8 plus 1/100 protease Inhibitor Cocktail (Sigma)) followed by centrifugation (15,000 g 15 minutes, 4° C. ). The supernatants were boiled after addition of SDS-sample buffer (5% glycerol, 2% SDS, 62.5 mM Tris-HCl pH 6.8, 2% 2-mercaptoethanol, 0.01% bromophenol blue), separated on 10% SDS-polyacrylamide gel and transferred to nitrocellulose membranes (Schleicher and Schuell, Keene). The membranes were incubated for 1 hour in TBS-T (25 mmol/L Tris-base, 150 mmol/L NaCl, 0.05%,Tween 20, pH 7.4) containing 5% (wt/vol) nonfat dry milk to block nonspecific antibody binding and then incubated with horseradish peroxidase (HRP)-goat anti mouse IgM antibody (115-035-020, 1:5000, Jackson). Antibody-labeled proteins were detected by enhanced chemiluminescence (ECL) substrate on Kodak film.

In Situ Hybridization

WT and IgM^{-/-} embryos (12.5 dpc) and their extraembryonic tissues were fixed in buffered 10% formalin at 4° C. for 16 hours and processed for paraffin embedding. The 5 μm thick paraffin sections were prepared and mounted on TESPA subbed SuperFrost® Plus slides. To generate the probes, a consensus fragment of either mesenchymal Ig μHC or mesenchymal Ig δHC cDNAs was cloned into pCDNA3 vector (Stratagene) and pGEM-T (Promega), respectively. Sense and antisense riboprobes were transcribed in vitro (Promega kit) using [³⁵S]-labeled UTP. Radioactive In situ hybridization was performed according to previously published protocol⁷ with slight modifications. In brief, deparaffinized sections were heated in 2×SSC at 70° C. for 30 minutes, rinsed in distilled water and incubated with 10 μg/ml proteinase K in 0.2M Tris-HCl (pH7.4), 0.05 M EDTA at 37° C. for 20 minutes. After proteinase digestion, slides were postfixed in 10% formalin in PBS (20 minutes), quenched in 0.2% glycine (5 minutes), rinsed in distilled water, rapidly dehydrated through graded ethanol and air-dried. The hybridization mixture contained 50% formamide, 4×SSC (pH 8.0), 1×Denhardt's, 0.5 mg/ml herring sperm DNA, 0.25 mg/ml yeast RNA, 10 mM DTT, 10% dextran sulfate and 3×10⁴ cpm/μl of [³⁵S]-UTP-labeled riboprobe. After application of the hybridization mixture sections were covered with sheets of polypropylene film cut from autoclavable disposable bags and incubated in humidified chamber at 65° C. overnight. After hybridization covering film was floated off in 5×SSC with 10 mM DTT at 65° C. and slides were washed at high stringency: 2×SSC, 50% formamide, 10 mM DTT at 65° C. for 30 minutes and treated with RNAse A (10 μg/ml) for 30 minutes at 37° C. Slides were next washed in 2×SSC and 0.1×SSC (15 minutes each) at 37° C. Then slides were rapidly dehydrated through ascending ethanol and air-dried. For autoradiography slides were dipped in Kodak NTB-2 nuclear track emulsion diluted 1:1 with double-distilled water and exposed for 3 weeks in light-tight box containing desiccant at 4° C. Exposed slides were developed in Kodak D-19 developer, fixed in Kodak fixer and counterstained with hematoxylin-eosin. Microphotographs were taken using Zeiss Axioscop-2 microscope equipped with Diagnostic Instruments Spot RT CCD camera.

Reverse Transcription Polymerase Chain Reaction (RT-PCR) Analysis

RT-PCR was performed on cDNAs obtained from the indicated cells and tissues. Total RNA was isolated from the above cells or tissues using either TriReagent (Molecular Research Center) or RNeasy Mini Kit (Qiagen, Valencia) in accordance to the manufacturer's instructions. To prevent genomic DNA contamination, samples were treated with DNAse (of Roche, or provided with the kit). Single strand cDNAs were then prepared using SuperScript™ reverse transcriptase (Invitrogen). Analysis of gene expression was done using PCR with ReadyMix™ PCR Master Mix (ABgene of Advanced Biotechnologies Ltd). The primers that were used are summarized in Table 1. Primers generated for heavy and light chains were designed to the constant region of the specific chain mentioned.

Rapid Amplification of cDNA Ends (RACE)

The 5′ end of the mesenchymal Ig μHC or mesenchymal Ig δHC transcripts were mapped using the FirstChoice® RNA Ligase-mediated Rapid Amplification of cDNA Ends (RLM-RACE) kit (Ambion, Austin), in accordance with manufacturer's instructions. RNA was isolated from either MBA-2.1 cells or IgM^{-/-} MEFs using RNeasy Mini Kit (Qiagen) according to manufacturer's instructions. Nested PCRs were used to amplify the 5′ end of the mesenchymal Ig μHC transcript. The 5′ RACE outer primer provided was used for the outer PCR reaction, together with the specific primer: 5′-CACGGCAGGTGTACACATTCAGGTTC-3′ (SEQ ID NO: 24); whereas the 5′ RACE inner primer provided, together with the specific primer:

5′-CGTGGCCTCGCAGATGAGTTTAGACTTG-3′ (SEQ ID NO: 25) were used for the inner PCR reaction. Nested PCRs were then used to amplify the 5′ end of the mesenchymal Ig δHC transcript. The 5′ RACE outer primer provided was used for the outer PCR reaction, together with the specific primer: 5′-GGATGTTCACAGTGAGGTTGC-3′ (SEQ ID NO: 26); whereas the 5′ RACE inner primer provided, together with the specific primer: 5′-AGTGACCTGGAGGACCATTG-3′ (SEQ ID NO: 27) were used for the inner PCR reaction. The 3′-end of the mesenchymal Ig δHC transcript was mapped using the same total RNAs. First strand cDNA was generated using a tagged oligo(dT) primer (GIBCO-BRL, Grand Island) followed by RNAse-H reaction. The cDNA was then used as a template for PCR performed with the universal amplification primer (UAP) provided and the specific primer: 5′-GCAACCTCACTGTGAACATCCTG-3′ (SEQ ID NO: 28). A second PCR was obtained with the same UAP primer and the specific primer: 5′-GCTTAATGCCAGCAAGAGCCTAG-3′ (SEQ ID NO: 29). The resultant PCR products were cloned into pGEM-T (Promega) and sequenced.

Statistical Analysis:

Student's paired t-test was used to evaluate the significance of differences between experimental groups.

### Results

Expression of Pre-BCR/BCR Components in Primary and Long-term Cultured Mesenchyme:

IgM Deficiency Results in Up-regulation of δ Chain mRNA

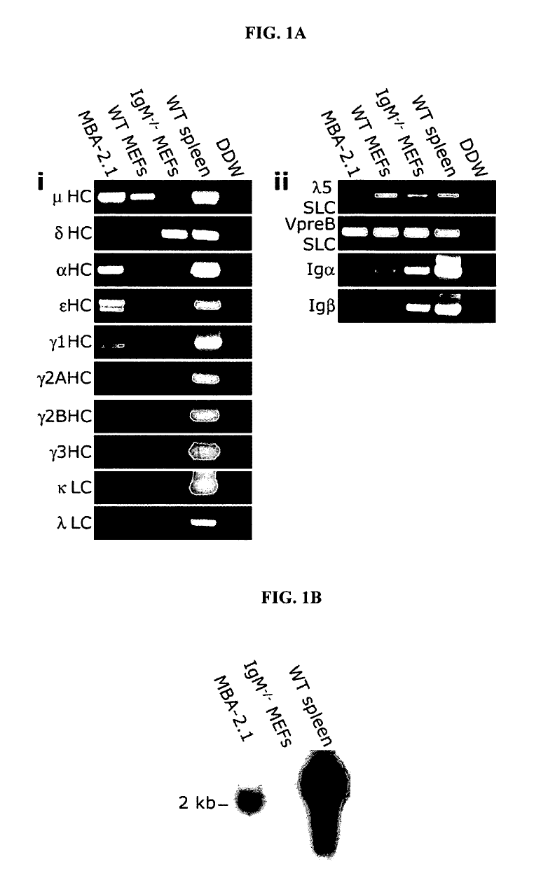

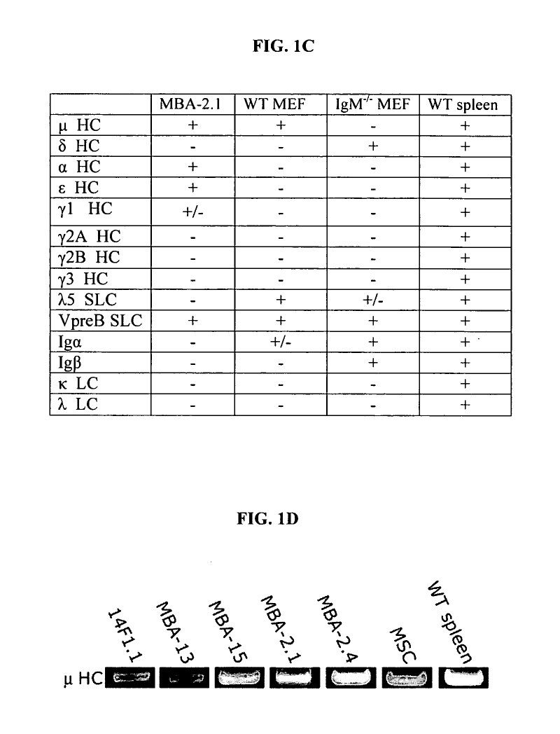

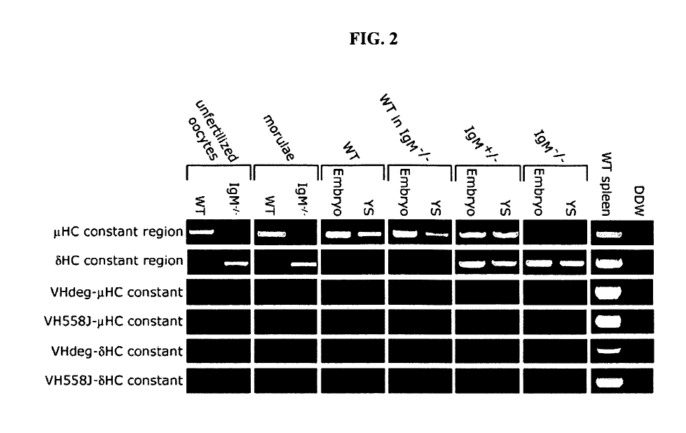

FIGS. 1A-1D show pre-BCR/BCR gene expression in mesenchyme: (1A) RT-PCR analysis of cDNAs obtained from the MBA-2.1 cell line, WT MEF and IgM^{-/-} MEF. (1Ai) Expression of the constant regions of the different Ig isotypes; (1Aii) Expression of SLCs and the pre-BCR accessory molecules. RNA from WT spleen and water (DDW) were used for positive and negative controls, respectively (the same controls were used for the RT-PCR analyses shown in FIG. 2). (1B) Northern blot analysis of Ig μHC transcripts: the amount of total RNA loaded in each lane: MBA-2.1 cells (40 μg); IgM^{-/-} MEFs (5 μg); and WT spleen (2 μg). (1C) A scheme of RT-PCR analysis from three independent experiments. +: expression, −: no expression, +/−: inconsistent (some cell batches were positive). (1D) μHC expression by several murine mesenchymal cell lines and primary mesenchymal stem cells (MSCs).

RT-PCR detected expression of Ig μHC mRNA in primary mouse embryo fibroblast (MEF) cell strains from 12.5 dpc embryos and in a cloned mouse bone marrow stromal cell line, MBA-2.1² (FIG. 1Ai). In contrast, MEF from IgM^{-/-} that serve as a negative control, had no such transcript. No expression of light chains was detected in WT MEF or MBA-2.1 cells, indicating that the μHC expression is not due to contamination of the mesenchymal cell cultures with lymphocytes. Northern blot analysis of mRNA from MBA-2.1 cells with a probe for μHC revealed a short transcript (˜2 KB) (FIG. 1B). Analysis of additional Ig isotypes indicated that δ chain is not found. Surprisingly, a μ to δ isotype exchange was observed in the IgM^{-/-} MEF (FIG. 1Ai). We further identified VpreB expression in the 3 cell types under study (FIG. 1Aii), as well as Igα, Igβ and λ5 that were, however, detected inconsistently in WT and IgM^{-/-} MEF and were not expressed in the MBA-2.1 cell line (FIG. 1C). Neither γ nor ε Ig isotypes were expressed in MEF, nor were κ and λ LCs (FIG. 1Ai). The μHC transcript was further detected in several murine mesenchymal cells lines that exhibit MSC functions^{4,6,8} as well as in primary bone marrow derived MSC (FIG. 1D).

Expression of Pre-BCR/BCR Components in the Early Embryo

To ascertain that the detection of Ig gene products in cultured mesenchyme was not an in vitro restricted phenomenon, the mRNAs were examined in embryonic tissues. FIG. 2 shows early embryonic expression of un-rearranged transcripts of Ig μHC or Ig δHC: RT-PCR analysis using primers of Ig μHC or Ig δHC constant regions and for rearranged versions of these transcripts. The primers used for the latter are VHdeg (a highly degenerate sense primer that amplifies the majority of the variable segment families) and VHJ558 a sense primer specific for the largest variable family J558 in Balb/c mice. The abbreviation “WT in IgM^{-/-}” refers to WT embryos that were transplanted at the morulae stage into IgM^{-/-} pseudo-pregnant recipient mothers.

Two different sets of primers were designed to enable RT-PCR detection of, and differentiation between, germline versus rearranged Ig μHC transcripts. Unfertilized oocytes were found to express an un-rearranged Ig μHC transcript whereas δHC was not detected (FIG. 2). Also, light chains expression was not observed (data not shown). Since the μHC transcripts were found in cells and tissues that are not expected to harbor such mRNAs, validity of the analysis was verified by examining tissues from μ chain deficient mice in which such transcripts were indeed absent (FIG. 2). In the IgM^{-/-} mouse oocytes the un-rearranged Ig μHC transcript was replaced by an un-rearranged Ig δHC transcript. Similarly, morulae from WT mice expressed un-rearranged Ig μHC mRNA whereas no δHC was detectable. The reverse was found in the IgM^{-/-} mice (FIG. 2). Although these results could imply that the expression of μ and δ chains is mutually exclusive, analysis of 11.5 dpc heterozygous (IgM^{+/-}) embryos revealed that both the un-rearranged Ig μHC and the Ig δHC transcripts were concomitantly detectable (FIG. 2). The expression of the Ig μHC mRNA in older 12.5 dpc WT and IgM^{-/-} embryos was investigated. The Ig μHC mRNA transcript was expressed both in the embryo proper and in the yolk sac while no Ig δHC expression was observed. In the IgM^{-/-} embryos and yolk sacs, expression of the δHC mRNA only was observed. The lack of expression of the B-cell marker, B220 (data not shown), or transcripts derived from Ig μ rearrangements, further supports the inference that the Ig μHC gene in WT embryos and the Ig δHC in IgM^{-/-} embryos are being expressed by non-lymphoid cells. To further assure that maternal lymphocytes do not account for detection of Ig transcripts in the embryonic tissues, RT-PCR analysis was performed using 12.5 dpc WT embryos that were transplanted, at the morulae stage, into IgM^{-/-} pseudo-pregnant recipient mothers. These embryos did express μHC (FIG. 2) thus providing strong evidence that Ig HC mRNAs that were detected are endogenous to the embryo.

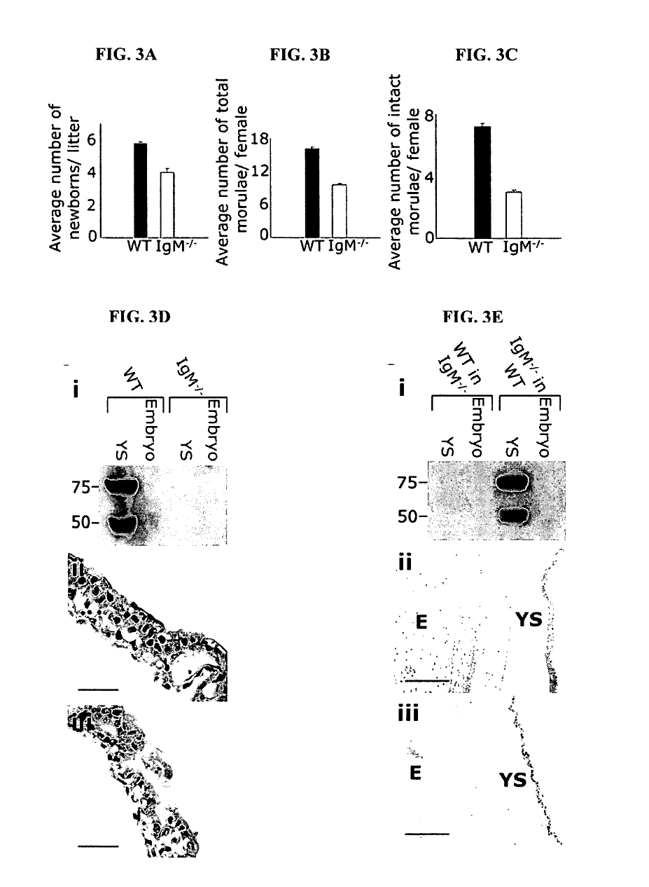

Although IgM^{-/-} mice exhibit normal B-cell development and maturation¹ the antibody repertoire in these animals is altered⁹. The question was therefore raised as to whether the lack of μHC mRNA would impact mesenchymal cell functions and early development. FIGS. 3A-3E show an increased incidence of defective morulae in IgM^{-/-} pregnancies and maternal origin of yolk sac IgM: Litter size and morulae properties: Litter size (3A) (Averages were derived from 120 deliveries in the IgM^{-/-} stock and 500 deliveries in the WT stock) and total number of morulae (3B), and number of intact morulae (3C) (a total of 65 IgM^{-/-} and 82 WT female mice). Values are means±standard error (p<0.0001). All differences shown are statistically significant. (3D) (i) Western analysis using anti-IgM antibody. immunohistochemcal staining using anti-IgM antibody of yolk sac from WT (ii) and IgM^{-/-} 12.5 dpc (days post coitus) embryos (iii). Original magnifications ×40, bar, 50 μM. (3E) (i) Western blot analysis using anti-IgM antibody. Immunohistochemical analysis using anti-IgM antibody was performed on sections from 12.5 dpc WT embryo transplanted into IgM^{-/-} pseudo-pregnant recipient mother (ii) and 12.5 dpc IgM^{-/-} embryo transplanted into WT pseudo-pregnant recipient mother. E—embryo, YS—yolk sac. Original magnifications: ×10, bar, 200 μM. The abbreviations “WT in IgM^{-/-}” refers to WT embryos that were transplanted at the morulae stage into IgM^{-/-} pseudo-pregnant recipient mothers, and the abbreviation “IgM^{-/-} in WT” refers to IgM^{-/-} embryos that were transplanted at the morulae stage into WT pseudo-pregnant recipient mothers.

In our animal stock, IgM^{-/-} mice, had smaller litter sizes than their WT counterparts (FIG. 3Ai). To examine early stages of development 2.5 dpc morulae from both IgM^{-/-} and WT mice were obtained. Four independent experiments were performed; in each experiment morulae were harvested from 12-20 female mice per group. The results of average total number of morulae per female are shown in FIG. 3Aii. IgM^{-/-} mice had an average number of 9.66±0.26 total morulae per female as compared with 16.2±0.28 per WT female. Furthermore, morulae were scored as having good developmental potential (being ‘intact’) if compacted and containing at least 4 cells, and up to 16 cells. IgM^{-/-} mice had an average number of 2.9±0.14 intact morulae per female as compared with 7.2±0.14 per WT female (FIG. 3Aiii). The reduced frequency of intact morulae imply a role for μ chain mRNA or protein in early development. Western blot analysis of protein extracts from 12.5 dpc embryo proper versus the yolk sac detected protein bands of 75 and 50 kDa only in WT yolk sac (FIG. 3Bi) visceral layer (FIG. 3Bii). This protein was maternally derived; 2.5 dpc WT morulae were transplanted into IgM^{-/-} pseudo-pregnant recipient mothers and vice versa. Subsequently, embryos were collected at 12.5 dpc. Both Western (FIG. 3Ci) and immunohistochemical (FIG. 3Cii,iii) analysis of tissues indicate that only yolk sacs derived from IgM^{-/-} embryos transplanted into WT pseudo-pregnant recipient mothers were IgM positive (FIG. 3Ci and iii).

Identification of the μHC and δHC mRNA Expressing Cells Within Mid-gestation Mouse Embryo

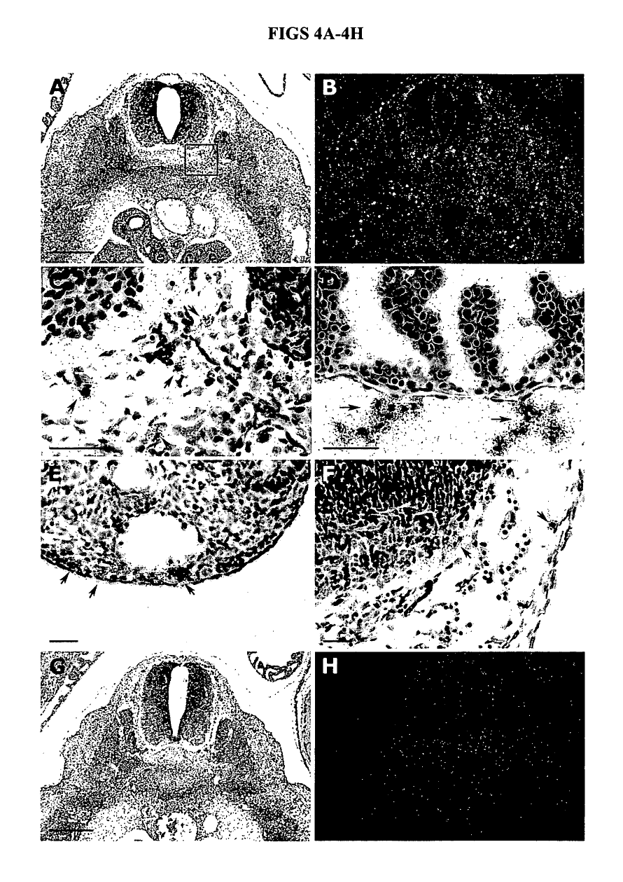

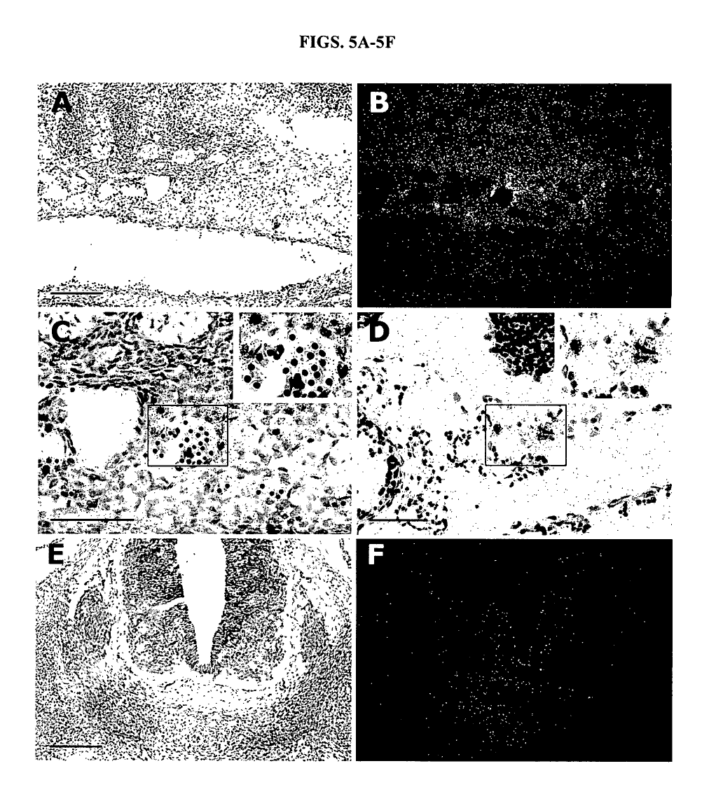

The nature of cells in mid-gestation that express BCR components was examined. Sections from both WT and IgM^{-/-} embryos were hybridized in situ with ³⁵S-labeled anti-sense RNA probes derived from the constant regions of either μHC (FIG. 4) or δHC (FIG. 5). In 12.5 dpc WT embryos, the positive cells expressing μHC were mesenchymal cells located in the loosely packed mesenchyme adjacent to the spinal cord (FIG. 4A-C), attached to the yolk sac (FIG. 4D) or similar cells in the proximity of blood vessels (FIG. 4E,F). No signal for μHC was detected in IgM^{-/-} embryos (FIG. 4G,H). FIGS. 4A-4H show that in situ hybridization localizes Ig μHC mRNA to embryonic mesenchyme: 35S-labeled anti-sense RNA probe derived from the constant region of Ig μHC was used to hybridize WT (A-F) and IgM^{-/-} (4G, 4H) 12.5 dpc embryos. Transverse sections of WT (4A,4B) and IgM^{-/-} (4G,4H) embryos stained with Hematoxylin-Eosin (4A, 4C, 4D, 4E, 4F) and dark field views of image 4A (4B) and 4G (4H) are shown, as well as an enlargement of the boxed area in image 4A (4C). Arrows point to representative positive cells. Original magnifications: 4A, 4B, 4G and 4H: ×10, bar, 200 μM; 4C, 4D: ×126 and 4E, 4F: ×63, bar, 20 μM.

In 12.5 dpc IgM^{-/}³¹ embryos, δHC positive cells were observed located in the proximity of blood vessels (FIG. 5A-C) or embedded within loose mesenchymal tissue (FIG. 5D). Thus, the in vivo identification of the Ig HC mRNAs expressing cells in mid-gestation embryos corroborates the in vitro detection of these mRNAs in mesenchyme. FIGS. 5A-5F show that in situ hybridization detects Ig δHC RNA expressing cells in IgM^{-/-} embryos: ³⁵S-labeled anti-sense RNA probe derived from the constant region of Ig δHC was used to hybridize IgM^{-/-} (5A-5D) and WT (5E, 5F) 12.5 dpc embryo sections. Bright field image of 5A and 5E are shown in 5B and 5D respectively. 5C and 5D are enlarged images of areas in (5A) and the insets in these images show more details of the boxed areas. Arrows indicate positive cells. Original magnifications: 5A, 5B, 5E and 5F ×20, bar, 100 μM. 5C: ×63, bar, 50 μM and ×90 (inset). 5D: ×40, bar, 50 μM and ×60 (inset).

Cloning and Structure Analysis of Ig μ and δHC Transcripts from Mesenchymal Cells

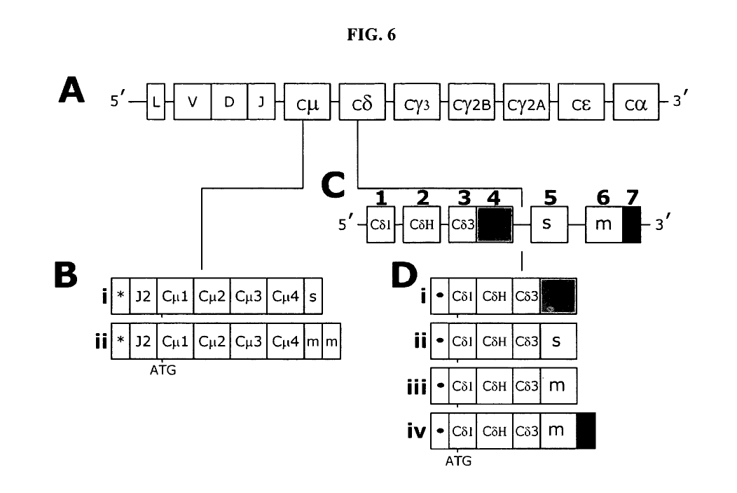

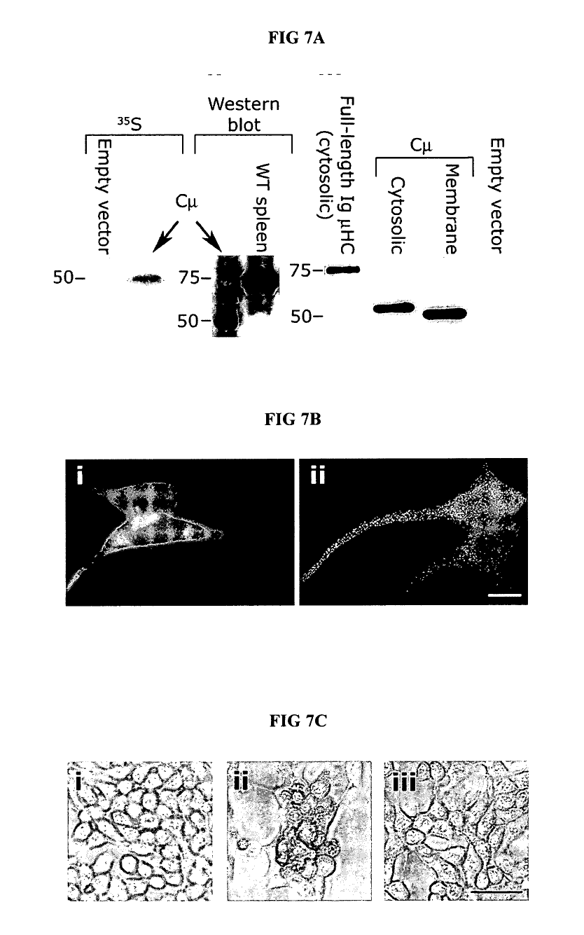

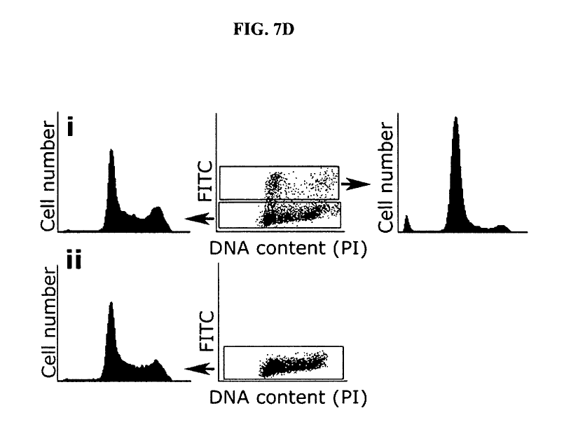

FIGS. 6A-6D show a schematic structure of μ and δ HC mRNAs cloned from WT and IgM^{-/-} mouse embryonic fibroblasts respectively:

(6A) The exon-intron structure of the entire immunoglobulin heavy chain (HC) locus. (6B) The mesenchymal truncated Ig μHC mRNA transcripts: the two isoforms comprise six identical exons. (*) indicates the unique genomic sequence-TTCTAAAGGGGTCTATGATAGTGTGAC (SEQ ID NO: 18) found on this mRNA, (J2) JH2 sequence, (Cμ1-Cμ4) represents the Ig μHC constant region exons, (s) represents secreted form sequence (isoform i); and (m) represents the two exons of the transmembrane domain (isoform ii). (6C). An enlargement of the δ constant HC region locus (1-7). (6D) Illustration of the mesenchymal truncated δHC transcripts: all four isoforms (i-iv) comprise the same first three exons, (·) indicates the unique genomic sequence-AAAGAATGGTATCAAAGGACAGTGCTTAGATCCAAGGTG (SEQ ID NO: 19), (Cδ1, CδH and Cδ3) represents the Ig δHC constant region exons (1-3). The four isoforms differ in their ending exons: isoform (i) possess exon (4) which does not have any known properties (colored in light gray); isoform (ii) possess exon (5) which has cytosolic features represents as (s); isoform (iii) possess exon (6) which contains a transmembrane domain, represents as (m) and isoform (iv) differs from isoform iii only in its additional non-coding 3′ end sequence (exon 7, colored in dark gray). L-leader sequence.

Rapid 5′ amplification of cDNA ends (RACE) using RNA derived from MBA-2.1 cells indicated that the mesenchymal Ig pHC transcript is an un-rearranged truncated form (FIG. 6B). A unique 5′ UTR is found in the mRNA that is homologous to a part of the μ switch region D-q52. Down stream to this 5′ UTR, the clone encodes the complete 4 exons of the Ig μHC constant region. Thus, this mRNA is a hybrid transcript that includes some exons from previously characterized genes. Both cytosolic and membrane type of transcript were cloned from the stromal cell line (FIG. 6B). The mesenchymal from of δHC lacks the variable segments that are upstream to the μHC constant region in the Ig locus (FIG. 6A). The MEF form of δHC consists of only the C region of the lymphoid form. The DNA sequence is composed of two C region domains, Cδ1 and Cδ3, separated by the CδH hinge domain. A 5′ UTR stretch of 39 bases is present upstream to the described C region (FIG. 6D and Table 1), which is homologous to a part of the μ switch region D-q52. Four distinctive 3′ ends that generate four mRNA isoforms of the mesenchymal truncated δ were isolated (FIG. 6D). Thus, mesenchyme expresses truncated forms of μ and δ HCs that consist of the C region only i.e. Cμ and Cδ. Since both transcripts contained in-frame ATGs (FIG. 6B,D) proteins could potentially be encoded.

TABLE 1
A summary of primers used in RT-PCR procedures

| Gene | Sense | Anti-sense |
|---|---|---|
| μHC | 5′-TAGGTTCAGTTGCTCACGAG | 5′-TGACCATCGAGAACAAAGG |
| | (SEQ ID NO: 30) | (SEQ ID NO: 31) |
| δHC | 5′-CTCCTCTCAGAGTGCAAAGCC | 5′-GGATGTTCACAGTGAGGTTGC |
| | (SEQ ID NO: 32) | (SEQ ID NO: 33) |
| αHC | 5′CATGAGCAGCCAGTTAACCCTG | 5′-ATGCAGCCATCGCACCAGCAC |
| | (SEQ ID NO: 34) | (SEQ ID NO: 35) |
| εHC | 5′GACTCCCTGAACATGAGCACTG | 5′-GGTACTGTGCTGGCTGTTTGAG |
| | (SEQ ID NO: 36) | (SEQ ID NO: 37) |
| γ1HC | 5′CTGGAGTCTGACCTCTACACTCTG | 5′CAGGTCAGACTGACTTTATCCTTG |
| | (SEQ ID NO: 38) | (SEQ ID NO: 39) |
| γ2AHC | 5′GATGTCTGTGCTGAGGCCCAGG | 5′-GGAAGCTCTTCTGATCCCAGAG |
| | (SEQ ID NO: 40) | (SEQ ID NO: 41) |
| γ2BHC | 5′GAGTCAGTGACTGTGACTTGGAAC | 5′-ACCAGGCAAGTGAGACTGAC |
| | (SEQ ID NO: 42) | (SEQ ID NO: 43) |
| γ3 HC | 5′-CTGGCTGCAGTGACACATCT | 5′-GGTGGTTATGGAGAGCCTCA |
| | (SEQ ID NO: 44) | (SEQ ID NO: 45) |
| λ5 SLC | 5′-TGGGGTTTGGCTACACAGAT | 5′-CCCACCACCAAAGACATACC |
| | (SEQ ID NO: 46) | (SEQ ID NO: 47) |
| VpreB | 5′-GTACCCTGAGCAACGACCAT | 5′-GTACCCTGAGCAACGACCAT |
| SLC | (SEQ ID NO: 48) | (SEQ ID NO: 49) |
| Igα | 5′-TGCCTCTCCTCCTCTTCTTG | 5′-TGATGATGCGGTTCTTGGTA |
| | (SEQ ID NO: 50) | (SEQ ID NO: 51) |
| Igβ | 5′-TCAGAAGAGGGACGCATTGTG | 5′-TTCAAGCCCTCATAGGTGTGA |
| | (SEQ ID NO: 52) | (SEQ ID NO: 53) |
| κLC | 5′-CTTGCAGATCTAGTCAGAGCC | 5′-CAATGGGTGAAGTTGATGTCTTG |
| | (SEQ ID NO: 54) | (SEQ ID NO: 55) |
| λLC | 5′CCAAGTCTTCGCCATCAGTCAC | 5′-GAACAGTCAGCACGGGACAAAC |
| | (SEQ ID NO: 56) | (SEQ ID NO: 57) |
| VH deg* | 5′SARGTNMAGCTGSAGSAGTCWGG | −ψ |
| | (SEQ ID NO: 58) | |
| VH | 5′-ATAGCAGGTGTCCACTCC | −ψ |
| J558** | (SEQ ID NO: 59) | |
| B220 | 5′-CAAAGTGACCCCTTACCTGCT | 5′-CTGACATTGGAGGTGTGTGT |
| | (SEQ ID NO: 60) | (SEQ ID NO: 61) |

*VH deg primer: a high degeneracy primer for mouse HC adopted from Chang 1992.
**VH J558 primer: a consensus signal sequence of the J558 V_{H} family, adopted from Ehlich 1994
ψ: Anti-sense primers were used depending on the gene of interest of either μ or δHCs.

Cμ mRNA Encodes a 50 KDa Protein that Localizes Intracellularly and Causes Growth Arrest

To examine whether the mesenchyme derived Cμ does encode a protein the cDNA was examined in an in vitro transcription/translation system. FIGS. 7A-7D show that a Cμ mRNA encodes a 50 kDa protein that causes growth arrest upon overexpression: (7A) Cμ protein synthesis in a cell free system translation/transcription system using ³⁵S-methionine as the radiolabel for the newly synthesized protein (i) and detection of the protein by antibodies to IgM μ chain, and protein expression of Cμ mRNA cloned in a mammalian expression vector and transfected into 293T cells (iii). (7B) Cellular localization of the cytosolic mesenchymal Cμ, or full-length Ig μHC. Immunofluorescence microscope analysis with anti-IgM antibodies was performed on cells transfected with cytosolic mesenchymal Ct (i), or cytosolic full-length Ig μHC (ii) in 293T cells. Original magnifications ×63, bar 20 μM. (7C) Phase-contrast images of 293T cells 24 hours after transfection with empty vector (i); cytosolic mesenchymal Cμ (ii) and cytosolic full-length Ig μHC (iii). Original magnifications ×20, bar, 100 μM. (7D) Overexpression of mesenchymal Cμ in 293T cells results in G1 arrest. (7Di) gating of cells stained positive and negative for IgM expression is shown in the middle panel. Left arrow shows cell cycle status of unstained 293T cells and the right arrow shows cell cycle status of positively stained 293T cells. (7Dii) Cell cycle pattern of 293T cells overexpressing empty vector.

FIG. 7A shows that this mRNA encodes a newly synthesized protein of approximately 50 kDa. An expression vector containing the mesenchymal Cμ transcript and the full-length Ig μHC were used to transfected 293T cells (FIG. 7B). Western blot analysis of extracts from Cμ transfected cells showed a protein band at about 50 kDa (FIG. Aiii). Whereas mesenchymal Cμ was found in a diffuse cytoplasmic staining, the full-length μHC chain was observed in punctate structures scattered throughout the cells (FIG. 7B). To get an insight as to the possible function of this truncated protein, the effects of overexpression in cultured cell lines was studied. The mesenchymal Cμ and the full-length μHC form B lymphocytes were compared following transfection of 293T cells. The overexpression of mesenchymal Cμ results in morphological changes in the cultured cells that were not seen with the full length μHC (FIG. 7Cii). Flow cytometric analysis showed that cells expressing the mesenchymal Cμ, exhibit a pronounced G1 arrest (FIG. 7Di, right panel). Cells negative for IgM expression (FIG. 7Di, left panel) or cells transfected with empty plasmid (FIG. 7Cii) have normal cell cycle distribution. In contrast, overexpression of full-length Ig μHC did not affect the cell cycle in a similar manner (data not shown).

### REFERENCES

Abbas et al (Eds.) (1994). Cellular and Molecular Immunology Chapter II, W. B. Saunders Co. (Philadelphia, USA).

Bagdy, G C., Heinrich, M C (1991) Vascular endothelial cells and hematopoiesis: regulation of gene expression in human vascular endothelial cells. Hemat Pathol 5,93-99

Barda-Saad, M, Shav-Tal, Y, Rozenszajn, A L, Cohen, M, Zauberman, A, Karmazyn, A, Parameswaran, R, Schori, H, Ashush, H, Ben-Nun, A, and Zipori, D (2002). The mesenchyme expresses T cell receptor mRNAs: relevance to cell growth control. Oncogene 21:2029-2036.

Bordignon C, Notarangelo L D, Nobili N, Ferrari G, Casorati G, Panina P, Mazzolari E, Maggioni D, Rossi C, Servida P, et al. (1995). Gene therapy in peripheral blood lymphocytes and bone marrow for ADA-immunodeficient patients. Science 270(5235):470-5

Bornemann, K. D., Brewer, J. W., Perez, E., Doerre, S., Sita, R., Corley, R. B. (1997) Secretion of soluble pre-B cell receptors by pre-B cells. J Immunol 15,2551-7

Cheng, A. M., Rowley, B. Pao, W. Hayday, A. Bolen, J. B. Pawson, T. (1995) Syk tyrosine kinase required for mouse viability and B-cell development. Nature 378,303

Ehlich, A., Martin, V., Müller, W. and Rajewsky, K. (1994) Analysis of the B cell progenitor compartment at the level of single cells. Curr Biol 4, 573-583

Fina, L., Molgaard, H. V., Robertson, D., Bradley, N. J., Monaghan, P., Delia, D., Sutherland, D. R., Baker, M. A.,and Greares, M. F. (1994) Expression of the CD34 gene in vascular endothelial cells. Blood 75, 2417-2426

Fleischman, R. A., Simpson, F., Gillardo, T., Jin, X., Perkins, S. (1995) Isolation of endothelial-like stromal cells that express kit ligand and support in vitro hematopoiesis. Exp Hematol 23, 1407-1416

Garcia Porrero, J. A., Godin, I. E., Dieterlen Lievre, F. (1995) Potential intraembryonic hemogenic sites at pre-liver stages in the mouse Anatomy and Embryology. Berlin 192, 425-435.

Gong, S., and Nussenzweig, M. C. (1996) Regulation of an early developmental checkpoint in the B cell pathway by Igβ. Science 272, 411-414

Hardy, R. R., Carmack, C. E., Shinton, S. A., Kemp, J. D. and Hayakawa, K. (1991) Resolution and characterization of pro-B and pre-pro B cell stages in normal mouse bone marrow. J Exp Med 173, 1213-1225

Horne, M. C., Roth, P. E., DeFranco, A. L. (1996) Assembly of the truncated immunoglobulin heavy chain Dμ into antigen receptor-like complexes in pre-B cells but not in B cells. Immunity 4,145-58

Ichihara, Y., Hayashida, H., Miyazawa, S., and Kurosawa, Y. (1989) Only DFL16, DSP2, and DQ52 gene families exist in mouse immunoglobulin heavy chain diversity gene loci, of which DFL16 and DSP2 originate from the same primordial DH gene. Eur J Immunol 19, 1849-1854

Kincade, P. (1981) Formation of B lymphocytes in fetal and adult life. Adv Immunol 31,177

Kitamura, D., Roes, J., Kuhn, R., and Rajewsky, K. A. (1991) B cell-deficient mouse by targeted disruption of the membrane exon of the immunoglobulin μ chain gene. Nature 350,423-6

Kitamura, D., Kudo, A., Schaal, S., Muller, W., Melchers, F., and Rajewsky, K. (1992a) A critical role of lambda 5 protein in B cell development. Cell 69,823-31

Kitamura, D., Rajewsky, K., (1992b) Targeted disruption of μ chain membrane exon causes loss of heavy-chain allelic exclusion. Nature 356,154-6

Lin, G., Finger, E., and Gutierrez-Ramos, J. (1995) Expression of CD34 in endothelial cells, hematopoietic progenitors and nervous cells in fetal and adult mouse tissues. Eur J Immunol 25, 1508-1515

Loffert, D., Ehlich, A., Muller, W., and Rajewsky, K. (1996) Surrogate light chain expression is required to establish immunoglobulin heavy chain allelic exclusion during early B cell development. Immunity 4 133-44

Lutz C, Ledermann B, Kosco-Vilbois M H, et al. IgD can largely substitute for loss of IgM function in B cells. Nature. 1998;393:797-801.

Melchers, F., Karasuyama, H., Haasner, D., Bauer, S., Kudo, A., Sakaguchi, N., Jameson, B. and Rolink, A. (1993) The surrogate light chain in B cell development. Immunol Today 14, 60-68

Melchers, F, Rolink, A (1999) B-Lymphocyte Development and Biology. In Paul, W E (Ed.), Fundamental Immunology. 4^{th} Ed. Lippincott-Raven Phila. 183-224

Mombaerts, P, Iacomini, J, Johnson, R, Herrup, K, Tonegawa, S, Papaioannou, V. (1992) RAG-1-deficient mice have no mature B and T lymphocytes. Cell 68, 869-877

Osmond, D. G. (1990) B cell development in the bone marrow. Semin Immunol 2, 173-180

Palacios, R., and Samaridis, J. (1992) Fetal liver pro-B and pre-B lymphocyte clones: expression of lymphoid-specific genes, surface markers, growth requirements, colonization of the bone marrow, and generation of B lymphocytes in vivo and in vitro. Mol Cell Biol 12,518-30

Rafii, S, Shapiro, F, Rimarachin, J, Nachman, R L, Ferris, B, Weksler, B, Moore, M A S, Asch, A S. (1994) Isolation and characterization of human bone marrow micro-vascular endothelial cells: hematopoietic progenitor cells adhesion. Blood 84, 10-19

Reth, M. G., and Alt, F. W. (1984) Novel immunoglobulin heavy chains are produced from DJH gene segment rearrangements in lymphoid cells. Nature 312,418-23

Rogers, J., Early, P., Carter, C., Calame, K., Bond, M., Hood, L., and Wall, R. (1980) Two mRNAs with different 3′ ends encode membrane-bound and secreted forms of immunoglobulin mu chain. Cell 20,303-12

Rolink, A. G., Winkler, T., Melchers, F. and Andersson, J. (2000) Precursor B cell receptor-dependent B cell proliferation and differentiation does not require the bone marrow or fetal liver environment. J Exp Med 191, 23-32

Shinkai, Y, Rathburn, G, Lam, K, Oltz, E M, Stewart V, Mendelsohn, M, Charron, J, Datta, M, Young, F, Stall, A M, Alt, R. (1992) RAG-2-deficient mice lack mature lymphocytes owing to inability to initiate V(D)J rearrangment. Cell 68, 855-867

Spanopoulou, E., C. A., Roman, L. M., Corcoran, M. S., Schlissel, D. P. Silver, D., Nemazee, M. C., Nussenzweig, S. A., Shinton, R. R., Hardy, and Baltimore. D. (1994) Functional immunoglobulin transgenes guide ordered B-cell differentiation in Rag-1-deficient mice. Genes Dev 8, 1030-1042

ten Boekel, E., Melchers, F., and Rolink, A. G. (1997) Changes in the V(H) gene repertoire of developing precursor B lymphocytes in mouse bone marrow mediated by the pre-B cell receptor. Immunity 7, 357-368

Tornberg, U. C., Bergqvist, I., Haury, M, Holmberg, D. (1998) Regulation of B lymphocyte development by the truncated immunoglobulin heavy chain protein Dμ J Exp Med 187,703-9

Torres, R. M., Flaswinkel, H., Reth, M., and Rajewsky. K. (1996) Aberrant B cell development and immune response in mice with a compromised BCR complex. Science 272,1804-1808

Turner, M., Mee, P J., Costello, P S, Williams, O, Price, A A, Duddy, L P, Furlong, M T, Geahlen, R. L., and Tybulewicz, V. L. J. (1995) Perinatal lethality and blocked B-cell development in mice lacking the tyrosine kinase Syk. Nature 378: 298-302

Wientroub S, Zipori D. (1996). “Stem Cell Culture” in: Principles of Bone Biology. J. Bilezikian, L. Raisz, G. Rodan, J. Markovac (eds). Academic Press, San Diego, pp 1267

Young, F., Ardman, B., Shinkai, Y., Lansford, R., Blackwell, T. K., Mendelsohn, M., Rolink, A., Melchers, F., and Alt. F. W. (1994) Influence of immunoglobulin heavy- and light-chain expression on B-cell differentiation. Genes Dev 8, 1043-1057

Zipori D (1989) Cultured stromal cell lines from hemopoietic tissues. In: Tavassoli M, ed, Blood Cell Formation: The Role of the Hemopoietic Microenvironment, Humana Press (Clifton, N.Y.), p. 287

Zipori D, Tamir M (1989). Stromal cells of hemopoietic origin. Int J Cell Cloning 7(5):281-91

Zipori D (1990). Stromal cells in tumor growth and regression. Cancer J 3: 164

What is claimed is:

1. An isolated polynucleotide molecule transcribed by immunoglobulin genes, said polynucleotide molecules lacking nucleic acid sequences that encode for V (variant) regions and said polynucleotide molecule comprising a 5′ intronic upstream sequence and nucleic acid sequences that encode a constant (C) domain.

2. The polynucleotide according to claim 1 encoded by an Ig μ heavy chain gene.

3. The polynucleotide according to claim 2 wherein the Ig p heavy chain gene comprises a nucleic acid sequence encoding a constant (Cμ) domain, and a 5′ intronic upstream sequence.

4. The polynucleotide according to claim 3 wherein the polynucleotide further comprising a nucleic acid sequence encoding a 5′ joining (J) region domain.

5. The polynucleotide according to claim 3 wherein the polynucleotide further comprising a nucleic acid sequence encoding 3′ secretory domain or a 3′ transmembrane domain.

6. The polynucleotide according to claim 3 wherein the polynucleotide is selected from a polynucleotide sequence set forth in any one of SEQ ID NOS: 9-11, SEQ ID NOS: 16-17 or a fragment thereof.

7. The polynucleotide according to claim 1 encoded by an Ig δ heavy chain gene.

8. The polynucleotide according to claim 7 wherein the Ig δ heavy chain gene comprises a nucleic acid sequence encoding a constant (C6) domain, and a 5′ intronic upstream sequence.

9. The polynucleotide according to claim 8 wherein the polynucleotide further comprising a nucleic acid sequence encoding 3′ secretory domain or a 3′ transmembrane domain.

10. The polynucleotide according to claim 8 wherein the polynucleotide is selected from a polynucleotide sequence set forth in any one of SEQ ID NOS: 12-15 or a fragment thereof.

11. An antisense nucleic acid molecule to the isolated polynucleotide molecule according to claim 1.

12. An RNAi nucleic acid molecule to the isolated polynucleotide molecule according to claim 1.

13. An expression vector comprising the polynucleotide molecules according to claim 1.

14. A host cell comprising the vector according to claim 13.

15. The host cell according to claim 14 wherein the cell is a mesenchymal cell.

16. An isolated polypeptide encoded by the polynucleotide according to claim 1.

17. An isolated Ig μ polypeptide according to claim 16 having an amino acid sequence set forth in any one of SEQ ID NOS: 1-3 or 7-8, or a fragment thereof.

18. An isolated Ig δ polypeptide according to claim 16 having an amino acid sequence set forth in any one of SEQ ID NOS: 1-3 or 7-8, or a fragment thereof.

19. An antibody raised to a polypeptide according to claim 16.

20. A pharmaceutical composition comprising as an active agent the polynucleotide molecule according to claim 1 and a pharmacologically acceptable carrier or excipient.

21. A pharmaceutical composition comprising as an active agent the host cell according to claim 14; and a pharmacologically acceptable carrier or excipient.

22. A pharmaceutical composition comprising as an active agent an isolated polypeptide according to claim 16; and a pharmacologically acceptable carrier or excipient.

23. A method of modulating mesenchymal intercellular interactions comprising the step of administering to a subject in need thereof a pharmaceutical composition according to claim 21 in an amount effective to induce mesenchymal intercellular interactions.

24. The method according to claim 23, wherein the polynucleotide comprises any one of SEQ ID NOS: 9-17.

25. The method according to claim 23, wherein the cells are of an autologous or allogeneic origin.

26. The method according to claim 23, wherein the method promotes or induces wound healing.

27. The method according to claim 23, wherein the method suppresses cell proliferation.

28. The method according to claim 27, wherein the method suppresses proliferation of cancer cells.

29. A method of modulating mesenchymal intercellular interactions comprising the step of administering to a subject in need thereof a pharmaceutical composition comprising a polypeptide according to claim 22 in an amount effective to induce mesenchymal intercellular interactions.

30. The method according to claim 29, wherein the method promotes or induces wound healing.

31. The method according to claim 30, wherein the method suppresses cell proliferation.

32. The method according to claim 31, wherein the method suppresses proliferation of cancer cells.
